(19) United States
(12) Patent Application Publication
(10) Pub. No.: US 2020/0196930 A1
(43) Pub. Date: Jun. 25, 2020

(54) OBSERVATION DEVICE, OBSERVATION METHOD, AND PROGRAM

(71) Applicant: SONY CORPORATION, TOKYO (JP)

(72) Inventors: GORO FUJITA, KANAGAWA (JP); TAKANORI FUKAZAWA, KANAGAWA (JP)

(21) Appl. No.: 16/640,570

(22) PCT Filed: Aug. 16, 2018
(86) PCT No.: PCT/JP2018/030432

§ 371 Date: Feb. 20, 2020

(30) Foreign Application Priority Data
Aug. 30, 2017 (JP) 2017-166009

(51) Int. Cl. A61B 5/1455; A61B 5/1459; G06T 7/00; G06T 7/77

(52) U.S. Cl. CPC A61B 5/14558; A61B 5/1459; G06T 2207/30004; G06T 7/77; G06T 7/0012

(57) ABSTRACT

An observation device according to an embodiment of the present technology includes a first polarization section, a second polarization section, a rotation control section, and a calculation section. The first polarization section irradiates a biological tissue with polarization light of a first polarization direction. The second polarization section extracts a polarization component of a second polarization direction that intersects with the first polarization direction, from beams of reflection light that are the polarization light reflected by the biological tissue. The rotation control section rotates each of the first polarization direction and the second polarization direction such that an intersection angle between the first polarization direction and the second polarization direction is maintained. The calculation section calculates biological tissue information related to the biological tissue on the basis of a change in intensity of the polarization component of the second polarization direction according to rotation operation performed by the rotation control section.

### TECHNICAL FIELD

The present technology relates to an observation device, an observation method, and a program that are applicable to observation of a biological tissue or the like.

### BACKGROUND ART

Conventionally, technologies of observing a biological tissue irradiated with polarized light have been developed. For example, Patent Literature 1 describes a polarization image measurement display system that displays a polarization property of a site of lesion or the like. According to Patent Literature 1, an imaging section captures 16 or more light intensity polarization images in different polarization states. A polarization conversion process section calculates a Mueller matrix of 4 rows×4 columns on the basis of the light intensity polarization images, and generates a polarization property image that shows a polarization property such as a depolarization ratio of a sample or a polarization ratio of light by using the Mueller matrix. When a combination of such polarization property images is displayed, it is possible for a doctor to identify presence or absence of a collagen fiber or the like (see paragraphs [0022], [0044] to [0046], [0094], FIGS. 7 and 15 or the like of Patent Literature 1).

### CITATION LIST

### Patent Literature

Patent Literature 1: Japanese Patent Application Laid-open No. 2015-33587

### DISCLOSURE OF INVENTION

### Technical Problem

Such biological tissue observation using polarization is expected to be applied to various situations such as surgery, medical diagnosis, and the like. Technologies capable of observing biological tissues in detail have been desired.

In view of the circumstances as described above, it is an object of the present technology to provide an observation device, an observation method, and a program that are capable of observing biological tissues in detail.

### Solution to Problem

In order to accomplish the above-mentioned object, an observation device according to an embodiment of the present technology includes a first polarization section, a second polarization section, a rotation control section, and a calculation section.

The first polarization section irradiates a biological tissue with polarization light of a first polarization direction.

The second polarization section extracts a polarization component of a second polarization direction that intersects with the first polarization direction, from beams of reflection light that are the polarization light reflected by the biological tissue.

The rotation control section rotates each of the first polarization direction and the second polarization direction such that an intersection angle between the first polarization direction and the second polarization direction is maintained.

The calculation section calculates biological tissue information related to the biological tissue on the basis of a change in intensity of the polarization component of the second polarization direction according to rotation operation performed by the rotation control section.

In this observation device, the biological tissue is irradiated with the polarization light of the first polarization direction. The polarization component of the second polarization direction that intersects with the first polarization direction is extracted from the beams of the reflection light reflected by the biological tissue. Each of the first polarization direction and the second polarization direction is rotated such that the intersection angle between the first polarization direction and the second polarization direction is maintained. The Biological tissue information is calculated on the basis of the change in the intensity of the polarization component according to the rotation operation. With this configuration, the biological tissue can be observed in detail.

The observation device may further include a detection section that detects, in accordance with the rotation operation, first intensity which is intensity of a polarization component of the second polarization direction extracted by the second polarization section. In this case, the calculation section may calculate, on the basis of the first intensity detected by the detection section, first intensity data related to a change in first intensity according to the rotation operation.

The calculation section may perform a fitting process using a predetermined function on the first intensity data and calculate the biological tissue information on the basis of a process result of the fitting process.

The biological tissue information may include identification information for identifying whether or not the biological tissue includes an optical anisotropic object.

The biological tissue information may include at least one of first information regarding an orientation direction of the optical anisotropic object or second information regarding orientation and anisotropy of the optical anisotropic object.

The calculation section may perform a fitting process using a predetermined periodic function, calculate the first information on the basis of phase information of the predetermined periodic function which is obtained as a process result of the fitting process, and calculate the second information on the basis of amplitude information of the periodic function.

The detection section may generate, in accordance with the rotation operation, an image signal of the biological tissue on the basis of the polarization component of the second polarization direction extracted by the second polarization section and detect the first intensity on the basis of the generated image signal.

The calculation section may set a plurality of target regions, into which an image constituted by the image signal is to be divided, and calculate the biological tissue information with respect to each of the plurality of target regions.

The observation device may further include a third polarization section that extracts the reflection light reflected by the biological tissue while maintaining a polarization state of the reflection light. In this case, the detection section may detect second intensity which is intensity of the reflection light extracted by the third polarization section.

The rotation control section may rotate the first polarization direction by a predetermined angle. In this case, the calculation section may calculate, on the basis of a change in the second intensity according to rotation of the first polarization direction by the predetermined angle, information regarding an orientation direction of an optical anisotropic object which is included in the biological tissue.

The rotation control section may rotate the first polarization direction by the predetermined angle on a basis of a predetermined state set on the basis of the change in the first intensity.

The predetermined angle may be ±90°.

The calculation section may determine a quadrant including the orientation direction among quadrants defined by a reference direction that is a reference of the orientation direction and an orthogonal direction orthogonal to the reference direction.

The calculation section may calculate an orientation angle between the orientation direction and the reference direction.

The observation device may further include: a fourth polarization section that emits non-polarized light to the biological tissue. In this case, the detection section may detect third intensity that is intensity of a polarization component of the second polarization direction extracted by the second polarization section from beams of the non-polarized light reflected by the biological tissue.

The rotation control section may rotate the second polarization direction by a predetermined angle. In this case, the calculation section may calculate, on the basis of a change in the third intensity according to rotation of the second polarization direction by the predetermined angle, information regarding an orientation direction of an optical anisotropic object which is included in the biological tissue.

The intersection angle may be an angle in a range of 90°±2°.

The observation device may be configured as an endoscope or a microscope.

An observation method according to an embodiment of the present technology is an observation method to be performed by a computer system and includes irradiating a biological tissue with polarization light of a first polarization direction.

A polarization component of a second polarization direction that intersects with the first polarization direction is extracted from beams of reflection light that are the polarization light reflected by the biological tissue.

Each of the first polarization direction and the second polarization direction is rotated such that an intersection angle between the first polarization direction and the second polarization direction is maintained.

Biological tissue information related to the biological tissue is calculated on the basis of a change in intensity of the polarization component of the second polarization direction according to rotation operation of the first polarization direction and the second polarization direction.

A program according to an embodiment of the present technology causes a computer system to execute the following steps.

A step of irradiating a biological tissue with polarization light of a first polarization direction.

A step of extracting a polarization component of a second polarization direction that intersects with the first polarization direction, from beams of reflection light that are the polarization light reflected by the biological tissue.

A step of rotating each of the first polarization direction and the second polarization direction such that an intersection angle between the first polarization direction and the second polarization direction is maintained.

A step of calculating biological tissue information related to the biological tissue on the basis of a change in intensity of the polarization component of the second polarization direction according to rotation operation of the first polarization direction and the second polarization direction.

### Advantageous Effects of Invention

As described above, in accordance with the present technology, it is possible to observe biological tissues in detail. It should be noted that the effects described here are not necessarily limitative and any effect described in the present disclosure may be provided.

### BRIEF DESCRIPTION OF DRAWINGS

FIG. 1 A diagram schematically showing a configuration example of an endoscopic device that is an observation device according to a first embodiment of the present technology.

FIG. 2 A schematic diagram showing an example of reflection by an observation target.

FIG. 3 A diagram showing specific examples of specular reflection.

FIG. 4 A schematic diagram showing examples of reflection caused inside an observation target.

FIG. 5 A schematic view for describing consideration regarding first intensity detected in a case of carrying out substantially crossed nicols observation of reflection light reflected by the anisotropic object.

FIG. 6 A graph showing the first intensity detected in a case of carrying out crossed nicols observation of the anisotropic object.

FIG. 7 A schematic view showing an example of crossed nicols observation.

FIG. 8 A diagram showing an example of a result of observation of crossed nicols observation.

FIG. 9 A schematic view showing an example of a result of observation of crossed nicols observation.

FIG. 10 A schematic view for describing an observation target.

FIG. 11 A schematic view showing an example of an image of the observation target imaged in crossed nicols observation.

FIG. 12 A flowchart showing an example of observation of a biological tissue.

FIG. 13 A diagram for describing an example of the process of calculating the biological tissue information on the basis of an image signal generated in crossed nicols observation.

FIG. 14 A diagram showing a specific example of process of calculating the biological tissue information shown in FIG. 13.

FIG. 15 A schematic view showing an example of an identification result of the anisotropic object according to crossed nicols observation.

FIG. 16 A schematic view showing an example of the biological tissue information calculated in crossed nicols observation.

FIG. 17 A diagram for describing a relation between an incident polarization angle θ and fiber directions in crossed nicols observation.

FIG. 18 A diagram for describing a relation between the incident polarization angle θ and the fiber directions in crossed nicols observation.

FIG. 19 A schematic view showing examples in a case of displaying the fiber directions by using information regarding the fiber directions of the anisotropic object which are calculated in crossed nicols observation.

FIG. 20 A schematic view showing an example of observation of the anisotropic object according to one nicol observation.

FIG. 21 A schematic view for describing consideration regarding second intensity detected in a case of carrying out one nicol observation of reflection light reflected by the anisotropic object.

FIG. 22 A schematic view for describing quadrants including fiber directions of the anisotropic object.

FIG. 23 A diagram showing an example of the first intensity detected in a case of carrying out crossed nicols observation of the anisotropic object.

FIG. 24 A diagram for describing an example of a determination process of the quadrants including the fiber directions.

FIG. 25 A flowchart showing an example of the determination process of the quadrants including the fiber directions.

FIG. 26 A schematic view showing an example of an image of the observation target imaged in one nicol observation.

FIG. 27 A diagram showing a process result of the determination process of the quadrants including the fiber directions.

FIG. 28 A schematic view showing another configuration example for performing one nicol observation.

FIG. 29 A result of detection of the fiber directions using one nicol observation.

FIG. 30 A diagram showing an example of a calculation process of the fiber directions using detection results according to crossed nicols observation and one nicol observation.

FIG. 31 A diagram for describing reflection in one nicol observation on an illumination side.

FIG. 32 A diagram showing an example of a threshold process regarding detection intensity of one nicol observation.

FIG. 33 A diagram showing a result of the threshold process using a first threshold.

FIG. 34 A diagram showing another result of the threshold process regarding the detection intensity of one nicol observation.

FIG. 35 A diagram showing an example of a result of observation of the fiber directions using one nicol observation which is shown as a comparative example.

FIG. 36 A diagram schematically showing a configuration example of an endoscopic device that is an imaging device according to another embodiment of the present technology.

### MODE(S) FOR CARRYING OUT THE INVENTION

Hereinafter, embodiments according to the present technology will be described with reference to the drawings.

### First Embodiment

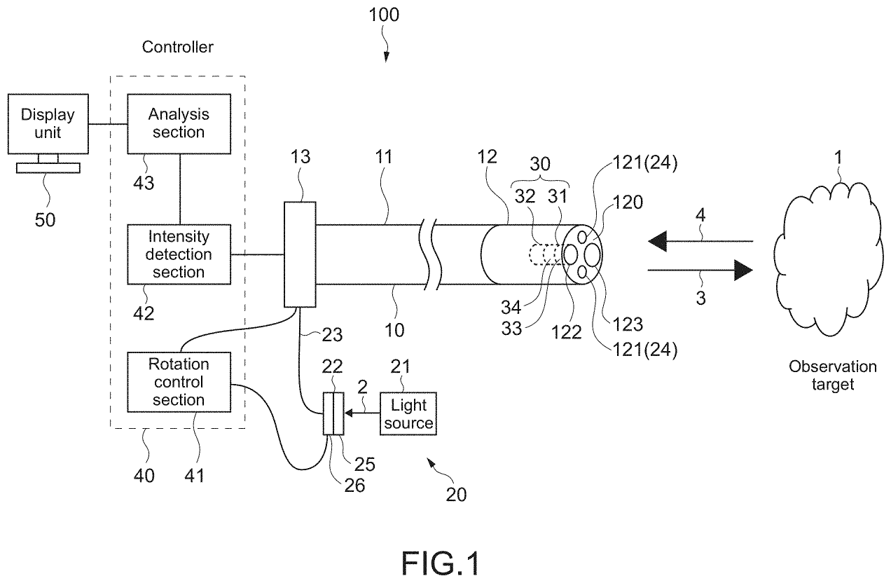

FIG. 1 is a diagram schematically showing a configuration example of an endoscopic device that is an observation device according to a first embodiment of the present technology. An endoscopic device 100 includes an insertion unit 10, an illumination system 20, an imaging system 30, a controller 40, and a display unit 50. The endoscopic device 100 is capable of observing an observation target 1 such as a site of lesion by inserting the insertion unit 10 into a mouth, an anus, or the like of a patient. In this embodiment, the observation target 1 is a biological tissue.

The insertion unit 10 includes a soft section 11, a tip section 12, and an operation section 13. The soft section 11 has a soft tubular structure. The diameter, length, and the like of the soft section 11 are not limited, and may be set as appropriate in accordance with the body shape of a patient, an insertion part of the patient such as a digestive tract or a trachea, or the like.

The tip section 12 is provided at one end of the soft section 11. The tip section 12 is inserted into the body of the patient, and is used for observation, treatment, or the like of the observation target 1. The tip section 12 includes a tip surface 120 that faces the observation target 1. The tip section 12 is bendable in such a manner that the tip surface 120 faces various directions.

As shown in FIG. 1, the tip surface 120 has illumination openings 121, an imaging opening 122, and a treatment tool outlet 123. Through the treatment tool outlet 123, a treatment tool such as forceps or a snare moves in and out. The specific configuration of the tip surface 120 is not limited. For example, the tip surface 120 may be appropriately provided with a nozzle or the like that is an outlet of water, air, or the like.

The operation section 13 is provided with an operation handle for adjusting the direction of the tip surface 120, and various kinds of connectors such as a video connector or an optical connector (they are not shown by the drawings). In addition, the operation section 13 may be appropriately provided with a switch or the like that is necessary to operate the insertion unit 10.

The illumination system 20 includes a light source 21, a first polarization element 22, a polarization maintaining fiber 23, and an illumination lens 24. The light source 21 is installed separately from the insertion unit 10, and emits illumination light 2 toward the first polarization element 22. In this embodiment, non-polarized light is used as the illumination light 2. The non-polarized light does not have a specific polarization direction. As the light source 21, it is possible to use a white light emitting diode (LED), a xenon lamp, or the like. Alternatively, any light source 21 capable of emitting non-polarized light can be used as appropriate.

The first polarization element 22 polarizes at least part of illumination light 2 emitted from the light source 21, in a first polarization direction. In other words, the first polarization element 22 generates linearly polarized light of the first polarization direction, from the illumination light 2 incident on the first polarization element 22.

For example, in a case where the non-polarized illumination light 2 is incident on the first polarization element 22, the first polarization element 22 extracts a polarization component that vibrates in the first polarization direction, from the non-polarized illumination light 2. As described above, polarization of the illumination light 2 in the first polarization direction includes extraction of the polarization component of the first polarization direction from the non-polarized illumination light 2.

In this embodiment, an optical element (liquid crystal polarizer) is used as the first polarization element 22. The optical element includes a polarizing plate 25 and a liquid crystal variable wave plate 26. The polarizing plate 25 has a predetermined polarization axis, and is disposed fixedly with respect to the light source 21. The liquid crystal variable wave plate 26 is disposed across the polarizing plate 25 from the light source 21. It should be noted that in FIG. 1, the polarization axis of the polarizing plate 25 is not shown for ease of explanation.

The polarizing plate 25 extracts linearly polarized light that vibrates in a direction parallel to the polarization axis of the polarizing plate 25, from the illumination light 2 incident on the polarizing plate 25. The polarization direction of the linearly polarized light that has been extracted is rotated by the liquid crystal variable wave plate 26, and then the linear polarization light is emitted. In other words, the linearly polarized light that has passed through the polarizing plate 25 and rotated by the liquid crystal variable wave plate 26 is the polarization light of the first polarization direction.

In addition, it is possible to arbitrarily set the first polarization direction by electrically controlling the liquid crystal variable wave plate 26. In other words, it is possible to generate linearly polarized light of any polarization direction by appropriately controlling a rotation angle of the linearly polarized light that has passed through the polarizing plate 25. In addition, when using the liquid crystal variable wave plate 26 rather than mechanically rotating the polarizing plate 25, it is possible to instantaneously change the first polarization direction, in other words, it is possible to quickly rotate the first polarization direction.

The specific configuration of the first polarization element 22 is not limited. For example, instead of the liquid crystal, it is possible to use an optical element using a transmissive ferroelectric substance such as PLZT. In addition, for example, an element capable of mechanically rotating the polarizing plate such as a wire grid polarizer or polarizing film may be used as the first polarization element 22. In addition, it is possible to appropriately configure the first polarization element 22 by using elements such as a polarizing plate or a wave plate.

The polarization maintaining fiber 23 is an optical fiber capable of transmitting light while substantially maintaining a polarization state of light. For example, the polarization maintaining fiber 23 is inserted into the operation section 13 from the first polarization element 22, passes through the inside of the soft section 11, and extends to the tip section 12. The polarization maintaining fiber 23 guides polarization light of the first polarization direction that has been emitted from the first polarization element 22, to the tip section 12 of the insertion unit 10 while substantially maintaining its polarization state. The specific configuration of the polarization maintaining fiber 23 is not limited. It is possible to appropriately use an optical fiber or the like capable of maintaining a polarization direction of linearly polarized light.

The illumination lenses 24 are disposed in the illumination openings 121 made in the tip surface 120 of the tip section 12. The illumination lens 24 magnifies the polarization light of the first polarization direction that has been passed through the polarization maintaining fiber 23, and emits the magnified light to the observation target 1. In FIG. 1, an arrow schematically represents polarization light 3 of the first polarization direction that is emitted from the illumination lenses 24. The specific configurations of the illumination lenses 24 are not limited. For example, any lenses capable of magnifying polarized illumination light may be used as the illumination lenses 24.

As described above, in the illumination system 20, the first polarization element 22 polarizes the illumination light 2 emitted from the light source 21 in the first polarization direction, and emits the polarized light to the observation target 1 via the polarization maintaining fiber 23 and the illumination lens 24. In this embodiment, the illumination system 20 corresponds to a first polarization section that irradiates a biological tissue with polarization light polarized in the first polarization direction.

The imaging system 30 includes a second polarization element 31 and an image sensor 31, and is disposed inside the tip section 12. In FIG. 1, dotted lines schematically represents the imaging system 30 (the second polarization element 31 and the image sensor 32) disposed inside the tip section 12.

The second polarization element 31 is disposed in the imaging opening 122. Reflection light 4 is incident on the second polarization element 31. The reflection light 4 is the polarization light 3 reflected by the observation target 1. In FIG. 1, an arrow schematically represents the reflection light 4 reflected by the observation target 1. It should be noted that sometimes the reflection light 4 may include polarization components in various polarization states.

Among beams of the reflection light 4 reflected by the observation target 1, the second polarization element 31 extracts a polarization component of a second polarization direction that intersects with the first polarization direction. In other words, the second polarization element 31 has a function of taking out the polarization component that vibrates in the second polarization direction from the reflection light 4 incident on the second polarization element 31.

In this embodiment, a liquid crystal polarizer including a liquid crystal variable wave plate 33 and a polarizing plate 34 is used as the second polarization element 31. As shown in FIG. 1, in the liquid crystal polarizer serving as the second polarization element 31, the liquid crystal variable wave plate 33 is disposed in such a manner that the liquid crystal variable wave plate 33 faces the observation target 1, and the polarizing plate 34 is disposed on a side opposite to the side where the liquid crystal variable wave plate 33 faces the observation target 1.

The reflection light 4 is incident on the liquid crystal variable wave plate 33. The liquid crystal variable wave plate 33 rotates the entire reflection light 4 in such a manner that a polarization component of the second polarization direction included in the reflection light 4 passes through the polarizing plate 34 in a subsequent stage.

For example, in a case where the second polarization direction is parallel to the polarization axis of the polarizing plate 34, the liquid crystal variable wave plate 33 transmits the reflection light 4 without rotating the reflection light 4. As a result, a polarization component that is included in the reflection light 4 and that is parallel to the polarization axis of the polarizing plate 34, that is, the polarization component of the second polarization direction passes through the polarizing plate 34, and is extracted. Alternatively, in a case where the second polarization direction is different from the polarization axis of the polarizing plate 34, the liquid crystal variable wave plate 33 rotates all the polarization components included in the reflection light 4 in such a manner that the second polarization direction becomes identical to the polarization axis of the polarizing plate 34 after the rotation. This makes it possible to extract the optical component of the second polarization direction.

In addition, it is possible to control the polarization component of the second polarization direction that is an extraction target, by controlling a rotation angle at the liquid crystal variable wave plate 33. For example, by appropriately setting the rotation angle at the liquid crystal variable wave plate 33, it is possible to extract a polarization component of a desired polarization direction (the second polarization direction) from the reflection light 4. It is also possible to quickly rotating the polarization direction (the second polarization direction).

The specific configuration of the second polarization element 31 is not limited. For example, instead of the liquid crystal, it is possible to use the optical element using the transmissive ferroelectric substance such as PLZT. In addition, for example, it is possible to use the element capable of mechanically rotating the wire grid polarizer, polarizing film, and the like. In addition, it is possible to appropriately configure the second polarization element 31 by using elements such as the polarizing plate and the wave plate. In this embodiment, the second polarization element 31 functions as a second polarization section.

The image sensor 32 is disposed across the second polarization element 31 from the observation target 1. In other words, the reflection light 4 is incident on the image sensor 32 from the observation target 1 via the second polarization element 31.

The image sensor 32 generates an image signal of the observation target 1 on the basis of the polarization component of the second polarization direction which is extracted by the second polarization element 31. The image signal is a signal capable of constituting an image, and includes a plurality of pixel signals each including luminance information. The image consisting of the image signal may be a color image, a black and white image, or the like. In addition, for example, the luminance information includes information such as a luminance value of each pixel, and RGB values indicating intensities of respective colors including red R, green G, and blue B of each pixel. The type, format, and the like of the image signal are not limited. Any format of the image signal may be used. The generated image signal is output to the controller 40.

As the image sensor 32, it is possible to use a charge coupled device (CCD) sensor, a complementary metal oxide semiconductor (CMOS) sensor, or the like, for example. As a matter of course, it is possible to use another type of sensor.

In addition, in this embodiment, the imaging system 30 is configured to be capable of removing the second polarization element 31 from the optical path of the reflection light 4. By removing the second polarization element 31 from the optical path of the reflection light 4, the reflection light 4 can be extracted without changing the polarization state of the reflection light 4. In this embodiment, a third polarization section is realized by removing the second polarization element 31 from the optical path of the reflection light 4.

The configuration for extracting the reflection light 4 while maintaining the polarization state of the reflection light 4 is not limited, any configuration may be used. That is, the method of realizing the third polarization section is not limited to the case of removing the second polarization element 31 from the optical path, and another method may be used. It should be noted that details of the case of extracting the reflection light 4 while maintaining the polarization state of the reflection light 4 will be described later with reference to FIG. 19 and the like.

The controller 40 includes hardware that is necessary for configuring a computer such as a CPU, ROM, RAM, and an HDD. An observation method according to the present technology is performed when the CPU loads a program into the RAM and executes the program according to the present technology. The program according to the present technology is recorded in the ROM or the like in advance. For example, the controller 40 can be implemented by any computer such as a personal computer (PC).

As shown in FIG. 1, in this embodiment, a rotation control section 41, an intensity detection section 42, and an analysis section 43 are configured as functional blocks when the CPU executes a predetermined program. As a matter of course, it is also possible to use dedicated hardware such as an integrated circuit (IC) to implement each of the blocks. The program is installed in the controller 40 via various kinds of recording media, for example. Alternatively, it is also possible to install the program via the Internet.

The rotation control section 41 is capable of rotating each of the first polarization direction and the second polarization direction. For example, the rotation control section 41 outputs respective control signals or the like to the first polarization element 22 and the second polarization element 31 for setting angles of the first and second polarization directions. This makes it possible to appropriately rotate each of the first polarization direction and the second polarization direction.

For example, by rotating the first polarization direction, it is possible to control the polarization direction of the polarization light to be emitted to the observation target 1. In addition, for example, it is possible to control the polarization direction of the polarization component extracted from the reflection light 4b y rotating the second polarization direction.

The rotation control section 41 rotates each of the first polarization direction and the second polarization direction such that an intersection angle between the first polarization direction and the second polarization direction is maintained. For example, the rotation control section 41 outputs respective control signals that instruct the first polarization element 22 and the second polarization element 31 to rotate the first polarization direction and the second polarization direction by predetermined angles. This makes it possible to perform rotation operation for rotating the first polarization direction and the second polarization direction by the predetermined angle while maintaining the intersection angle between the first polarization direction and the second polarization direction.

In addition, the rotation control section 41 rotates the first polarization direction and the second polarization direction in synchronization with each other. For example, the rotation control section 41 generates a synchronization signal such as a clock signal, and controls the first polarization element 22 and the second polarization element 31 in synchronization with each other on the basis of the synchronization signal. This makes it possible to rotate the first and second polarization directions at substantially the same timings.

It should be noted that the rotation control section 41 is capable of outputting the synchronization signal to the image sensor 32 or the like. By using the synchronization signal, the image sensor 32 is capable of generating an image signal of the observation target 1 in accordance with rotation operation performed by the rotation control section 41.

The intensity detection section 42 detects intensity of the polarization component of the second polarization direction that has been extracted by the second polarization element 31 in accordance with rotation operation performed by the rotation control section. Hereinafter, the intensity of the polarization component of the second polarization direction that has been extracted by the second polarization element 31 will be referred to as first intensity.

In this embodiment, the intensity detection section 42 detects the first intensity on the basis of an image signal of the observation target that have been generated by the image sensor 32. That is, the intensity detection section 42 acquires the image signal generated by the image sensor 32 in the respective states in which the first and second polarization directions have been rotated. Then, the intensity detection section 42 detects the first intensity with respect to each acquired image signal. Accordingly, the intensity detection section 42 is capable of detecting the first intensity in the respective states in which the first and second polarization directions have been rotated.

The intensity detection section 42 detects the first intensity for each pixel on the basis of, for example, information regarding the luminance value, the RGB value, and the like included in the luminance information of each pixel of the image signal. The detected first intensity is output to the analysis section 43. In this embodiment, the image sensor 32 and the intensity detection section 42 realize a detection section.

The analysis section 43 calculates biological tissue information related to the observation target 1 on the basis of the intensity of the polarization component of the second polarization direction according to the rotation operation performed by the rotation control section 41, i.e., a change in first intensity. In this embodiment, the analysis section 43 calculates data regarding the change in first intensity according to the rotation operation as first intensity data in accordance with the rotation operation on the basis of the detected first intensity.

Angles by which the first and second polarization directions have been rotated and the first intensity, for example, are stored in associated with each other as the first intensity data. Therefore, the first intensity data includes information indicating how the first intensity has changed in accordance with the rotation operation. The analysis section 43 analyzes the first intensity data to thereby calculate biological tissue information of the observation target 1.

In addition, the analysis section 43 analyzes the image signal of the observation target 1 generated by the image sensor 32. The analysis section 43 generates an intraoperative image of the observation target 1 on the basis of the analysis result of the image signal, the calculated biological tissue information, and the like. The intraoperative image is an image of the observation target 1 captured during surgery including observation, treatment, and the like performed by using the endoscopic device 100. In this embodiment, the analysis section 43 corresponds to a calculation section. Details of operation and the like of the analysis section 43 will be described later.

The display unit 50 displays the intraoperative image of the observation target 1 generated by the analysis section 43. For example, a display device such as a liquid crystal monitor is used as the display unit 50. For example, the display unit 50 is installed in a room where endoscopic observation is performed. This makes it possible for a doctor to perform observation and treatment while watching the intraoperative image displayed on the display unit 50. The specific configuration of the display unit 50 is not limited. For example, as the display unit 50, it is possible to use a head-mounted display (HMD) or the like capable of displaying the intraoperative image.

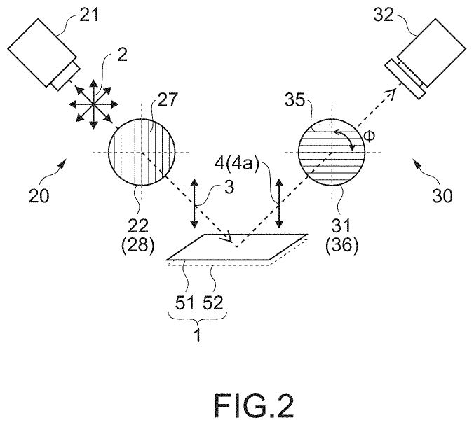

FIG. 2 is a schematic diagram showing an example of reflection by the observation target 1. With reference to FIG. 2, reflection by a surface 51 of the observation target 1 will be described. FIG. 2 schematically shows the light source 21 and the first polarization element 22 as the illumination system 20. Illustration of the polarization maintaining fiber 23 and the illumination lens 24 described with reference to FIG. 1 is omitted. In addition, as the imaging system 30, FIG. 2 schematically shows the second polarization element 31 and the image sensor 32.

To simplify the explanation, in FIG. 2, a polarizing plate 28 having a first polarization axis 27 represents the first polarization element 22 including the polarizing plate 25 and the liquid crystal variable wave plate 26. Among beams of the illumination light 2, the first polarization element 22 emits a polarization component of a direction parallel to the first polarization axis 27 as the polarization light 3 of the first polarization direction. This corresponds to a case where the liquid crystal variable wave plate 26 rotates the polarization direction of linearly polarized light extracted by the polarizing plate 25, and the linearly polarized light is emitted as the polarization light 3 of the first polarization direction.

In addition, a polarizing plate 36 having a second polarization axis 35 represents the second polarization element 31 including the polarizing plate 34 and the liquid crystal variable wave plate 33. The second polarization element 31 extracts a polarization component parallel to the second polarization axis 35 as a polarization component of the second polarization direction. This corresponds to a case where the liquid crystal variable wave plate 33 rotates reflection light 4 in such a manner that the polarization component of the second polarization direction passes through the polarizing plate 34.

The first and second polarization directions are rotated by electrically controlling the liquid crystal variable wave plates 26 and 33 when the polarizing plates 28 and 36 shown in FIG. 2 are rotated. It should be noted that the polarizing plates 28 and 36 are installed as the structural elements that are schematically shown in FIG. 2, that is, the first polarization element 22 and the second polarization element 31. In addition, mechanisms for physically rotating them are also included in the configurations of the first and second polarization sections according to the present technology.

In the example shown in FIG. 2, an intersection angle Φ between the first and second polarization directions is set to approximately 90 degrees, and the first and second polarization directions establish a substantially crossed nicols relation.

As shown in FIG. 2, in the illumination system 20, the light source 21 emits the non-polarized illumination light 2. Among beams of the illumination light 2, the first polarization element 22 extracts a polarization component of a direction parallel to the first polarization axis 27 as the polarization light 3 of the first polarization direction. The extracted polarization light 3 is emitted toward the observation target 1.

Part of the polarization light 3 incident on the observation target 1 is reflected near the surface 51 of the observation target 1. With respect to the reflection near the surface 51 of the observation target 1, a polarization state of light hardly changes from a polarization state of light incident on a reflection surface (the surface 51 of the observation target 1). This means that, the polarization state is maintained before and after the reflection.

Therefore, as shown in FIG. 2, reflection light 4a reflected near the surface 51 of the observation target 1 proceeds to the imaging system as linearly polarized light that maintains the first polarization direction but is affected by properties of the vicinity of the surface of the observation target. It should be noted that the other portions of the polarization light 3 incident on the observation target 1 are diffused/scattered through an inside 52 of the observation target 1 and reflected while their polarization directions are randomized due to multiple reflection.

The reflection light 4a polarized in the first polarization direction is incident on the second polarization element 31 of the imaging system 30. Since the first and second polarization directions establish the substantially crossed nicols relation, a polarization plane of the reflection light 4a polarized in the first polarization direction is substantially kept by the surface reflection. Therefore, the reflection light 4a hardly passes through the second polarization element 31, and most of the reflection light 4a are absorbed/reflected by the second polarization element 31. As a result, the reflection light 4a reflected near the surface 51 of the observation target 1 is hardly received by the image sensor 32 in the subsequent stage after the second polarization element 31.

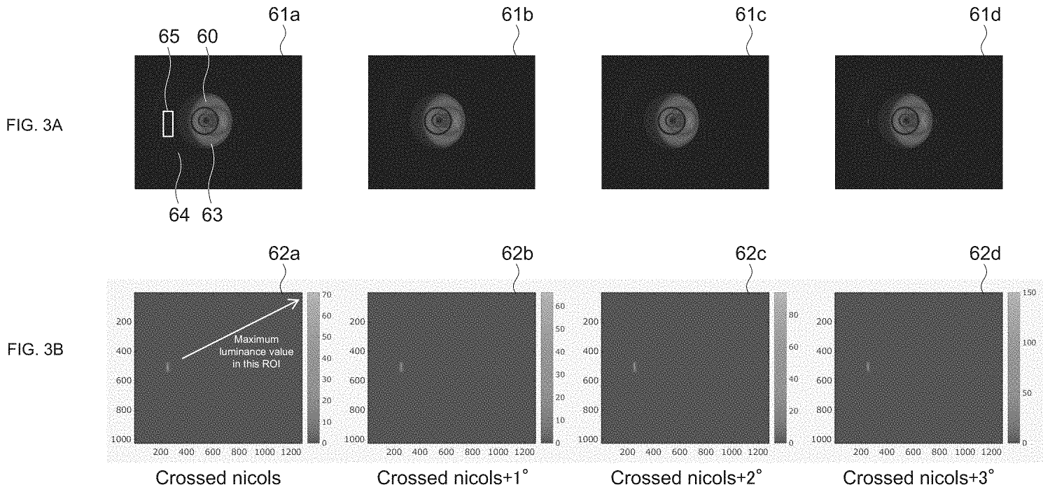

FIG. 3 is a diagram showing specific examples of specular reflection. FIG. 3A shows images 61a to 61d of a level 60 captured via the second polarization element 31 in a cases where an intersection angle Φ of the first and second polarization directions Φ is 90°, 91°, 92°, and 93°. FIG. 3B shows maps 62a to 62d showing reflection light intensity distributions with respect to the images 61a to 61d.

The level 60 includes a cylindrical bubble tube 63 at its center, and includes a metal frame 64 around the cylindrical bubble tube 63. The images 61a to 61d of the level 60 show images of the level 60 by reflection light diffusely reflected by the cylindrical bubble tube 63 and reflection light specularly reflected by the metal frame 64. Each of the images has been captured in a near-crossed nicols state. Therefore, the reflection light specularly reflected by the metal surface of the metal frame 64 is hardly received, and the metal frame 64 is displayed darkly.

The maps 62a to 62d shown in FIG. 3B show luminance distributions of gray scale luminance values in an analysis region (region of interest (ROI) 65). The ROI 65 is displayed in the image 61a. A vertical axis and a horizontal axis of each map correspond to the number of vertical and horizontal pixels in each image of the level. Gray scale bars represent luminance values in the ROI 65. The ROI 65 is set in such a manner that the ROI 65 is disposed on a boundary between the cylindrical bubble tube 63 and the metal frame 64.

In ideal crossed nicols observation, a specular reflection component is zero. In practice, some specular reflection components remain because of attenuation (extinction ratio) of polarization components parallel to the polarization axis of the polarizing plate, wavelength dependency of the polarizing plate, an incident angle on a subject (the observation target 1), deviation from an orthogonal state, or the like. For example, in the map 62a of a crossed nicols state where the intersection angle Φ is 90°, a slight specular reflection component remains in the ROI. With respect to the map 62a, the maximum luminance value in the ROI 65 is 71.

In a case where the intersection angle Φ between the first and second polarization directions deviates from the crossed nicols state (Φ=90°) by 1° (the map 62b), the maximum luminance value in the ROI 65 is 66. In a similar way, in a case where the intersection angle Φ deviates by 2° (the map 62c), the maximum luminance value in the ROI 65 is 94. In a case where the intersection angle Φ deviates by 3° (the map 62d), the maximum luminance value in the ROI 65 is 150. It should be noted that the maximum luminance values of the respective maps correspond to maximum values (brightest values) of the respective gray scale bars.

As described above, when the intersection angle θ between the first and second polarization directions deviates from the crossed nicols state by 3° more, the number of specular reflection components included in the reflection light 4a is suddenly increased. For example, the specular reflection components may be a cause of halation, reflected glare of illumination light (polarization light 3), or the like when the observation target 1 is observed. In addition, there is a possibility that the specular reflection component causes noise at a time of crossed nicols observation. Therefore, in a case where the intersection angle Φ deviates from the crossed nicols state by 3° or more, there is a possibility that effects of the reflected glare of illumination light or the like increases.

In this embodiment, the intersection angle Φ between the first and second polarization directions is set to an angle in a range of 90°±2°. When the intersection angle Φ is set to the range of 90°±2°, it is possible to sufficiently attenuate the specular reflection component, and it is possible to sufficiently attenuate the reflected glare of illumination light. A surface reflection component of a biological tissue is considered to be smaller than the specular reflection component of the metal material. Therefore, it is possible to accurately observe the observation target 1, and this makes it possible to sufficiently support observation of the biological tissue.

It should be noted that the range of the intersection angle Φ between the first and second polarization directions is not limited. The range of the intersection angle Φ may be appropriately set in a range capable of achieving acceptable observation accuracy. For example, the intersection angle Φ may be set to an angle in a range wider than 90°±2° such as 90°±5° or 90°±10°. For example, it is possible to appropriately set the intersection angle Φ in accordance with the type of observation target 1 and characteristics of the illumination system 20 and the imaging system 30.

A method of setting the intersection angle Φ between the first and second polarization directions to a desired value such as 90°±2° is not limited. For example, the intersection angle Φ may be set on the basis of a polarization component of the first polarization direction included in the reflection light 4a, that is, the specular reflection component.

For example, in FIG. 2, a sample including a metal surface with strong specular reflectivity is used as the observation target 1. First, the first polarization axis 27 of the first polarization element 22 is fixed, and illumination light (the polarization light 3) is emitted to the metal surface. The reflection light 4a polarized in the first polarization direction is emitted from the metal surface, and is incident on the second polarization element. Here, the second polarization axis 35 of the second polarization element 31 is rotated, and a total amount of light received by the image sensor 32 is detected.

For example, in a case where the first polarization direction is parallel to the second polarization axis 35, the reflection light 4a polarized in the first polarization direction substantially passes through the second polarization element 31, and the total amount of light received by the image sensor 32 becomes maximum. Accordingly, it is possible to set the intersection angle Φ between the first and second polarization directions to 90° by rotating the second polarization axis 35 by 90° on the basis of the angle at which the total amount of light is maximum. As a matter of course, it is also possible to set the intersection angle Φ on the basis of an angle at which the total amount of light is minimum. In addition, it is possible to use any method capable of setting the intersection angle Φ.

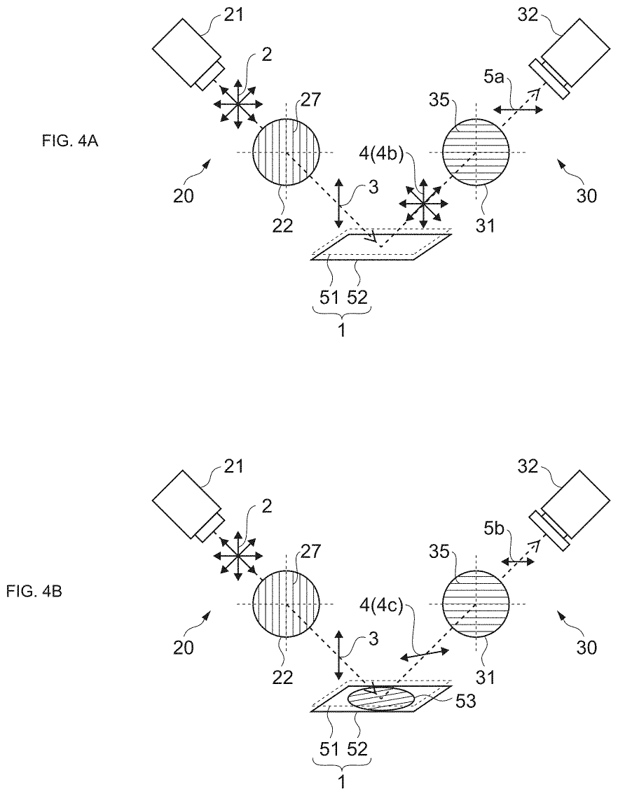

FIG. 4 is a schematic diagram showing an example of reflection caused in the inside 52 of the observation target 1. In FIGS. 4A and 4B, the first polarization element 22 and the second polarization element 31 are disposed so as to establish the substantially crossed nicols relation.

As shown in FIG. 4, the polarization light 3 of the first polarization direction emitted from the illumination system 20 is incident on the observation target 1. Part of the polarization light 3 incident on the observation target 1 is specularly reflected by the surface 51 of the observation target 1, and the other portions of the polarization light 3 are incident on the inside 52 of the observation target 1.

The inside 52 of the observation target 1 includes various kinds of biological tissues such as fat and muscle. The polarization light 3 is diffused, scattered, or absorbed, or a polarization direction of the polarization light 3 is rotated in accordance with optical characteristics of respective biological tissues. As a result, as shown in FIG. 4A, reflection light 4b multiply scattered in the inside 52 of the observation target 1 includes polarization components of various polarization directions.

The reflection light 4b reflected in the inside 52 of the observation target 1 is incident on the second polarization element 31. The second polarization element 31 extracts a polarization component of the reflection light 4b parallel to the second polarization axis 35 as a polarization component 5a of the second polarization direction. The extracted polarization component 5a is incident on the image sensor 32.

FIG. 4B is a schematic direction showing a case where the polarization light 3 of the first polarization direction is incident on an anisotropic object 53 in the inside 52 of the observation target 1. Here, for example, the anisotropic object 53 is an optically anisotropic biological tissue. Examples of the anisotropic object 53 of the biological tissue include muscle fibers of muscle, collagen fibers in cartilage such as a meniscus, and a nerve fascicles that are bundles of nerve fibers. As a matter of course, the present technology is not limited thereto. The present technology is applicable to any optically anisotropic tissue and the like. In this embodiment, the anisotropic object 53 corresponds to an optical anisotropic object.

For example, when the linearly polarized light is emitted to the anisotropic object 53, the polarization state changes in accordance with the optical characteristics of the anisotropic object 53. For example, due to optical rotation of the anisotropic object 53, a polarization direction of the linearly polarized light is rotated. In addition, due to circular dichroism of the anisotropic object 53, some polarization components of the linearly polarized light are absorbed and the linearly polarized light is polarized as elliptically polarized light. As a result, the anisotropic object 53 emits reflection light 4c in the polarization state different from that of the linearly polarized light emitted to the anisotropic object 53.

In addition, the polarization states of the reflection light 4c such as the polarization direction and ellipticity change in accordance with the polarization direction of the linearly polarized light that has been emitted. In other words, the polarization state, intensity, and the like of the reflection light 4c change in accordance with optical characteristics of the anisotropic object 53 and the polarization direction of the linearly polarized light emitted to the anisotropic object 53.

As shown in FIG. 4B, the polarization light 3 of the first polarization direction is emitted to the anisotropic object 53. The anisotropic object 53 emits the reflection light 4c whose polarization state has been changed. It should be noted that FIG. 4B schematically shows the reflection light 4c as the linearly polarized light. However, the present technology is not limited thereto. Sometimes elliptically polarized light or the like may be emitted as the reflection light 4c.

The reflection light 4c reflected by the anisotropic object 53 is incident on the second polarization element 31. The second polarization element 31 extracts a polarization component 5b of the second polarization direction among polarization components included in the reflection light 4c. The extracted polarization component 5b is emitted toward the image sensor 32.

When extracting the polarization component 5b, the second polarization element 31 reflects/absorbs a polarization component of the reflection light 4c that is orthogonal to the second polarization direction. Therefore, intensity (amount of light) of the extracted polarization component 5b varies in a manner that depends on the polarization state of the reflection light 4c polarized by the anisotropic object 53. It should be noted that in FIG. 4B, the intensity of the polarization component 5b is indicated by a length of an arrow representing the polarization component 5b.

Here, it is assumed that the first and second polarization directions are rotated while maintaining the crossed nicols relation. In this case, a polarization direction (the first polarization direction) of linearly polarized light emitted to the anisotropic object 53, and a polarization direction (the second polarization direction) of the polarization component 5b extracted by the second polarization element 31 change. Therefore, intensity of the polarization component 5b extracted by the second polarization element 31 changes. As described above, in the crossed nicols observation, the intensity of transmitted light (the polarization component 5b) that has passed through the second polarization element 31 is changed with rotation of the first and second polarization directions.

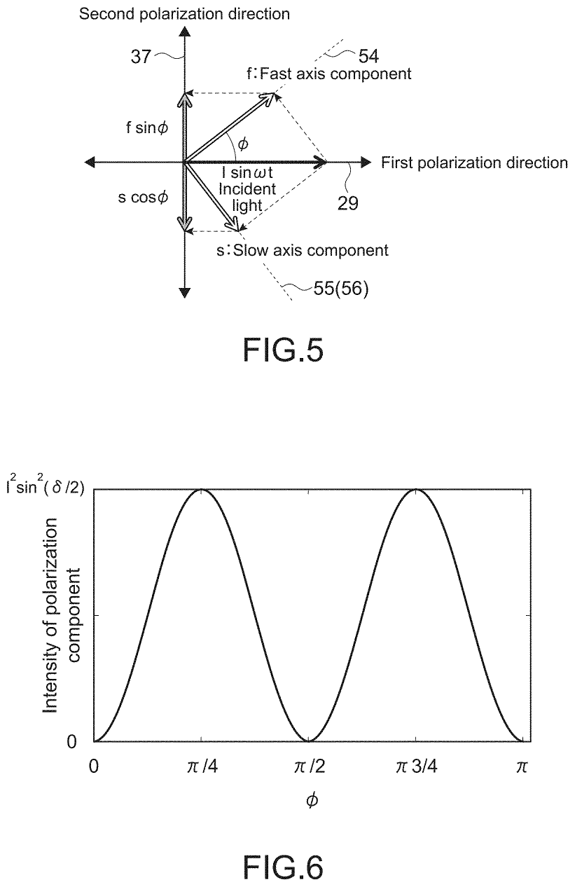

The inventor of the present technology has considered the first intensity detected in a case of carrying out substantially crossed nicols observation of reflection light reflected by the anisotropic object 53 as follows. FIG. 5 is a schematic view for describing the consideration. FIG. 5 schematically shows each of the polarization directions such that the first polarization direction 29 is the horizontal direction and the second polarization direction 37 orthogonal to the first polarization direction 29 is the vertical direction.

In general, an optically anisotropic object (the anisotropic object 53) includes a fast axis 54 and a slow axis 55. In the anisotropic object 53, the velocity of light travelling along the slow axis 55 is lower than that of light travelling along the fast axis 54. Therefore, the phase of light travelling along the slow axis 55 is delayed from the phase of light travelling along the fast axis 54. As described above, the anisotropic object 53 undergoes double refraction which is propagation of light divided into two light beams.

FIG. 5 schematically shows the fast axis 54 and the slow axis 55 orthogonal to each other. Hereinafter, the description will be given assuming that a direction parallel to the slow axis 55 is a fiber direction 56 of the anisotropic object 53. In addition, light absorption at the anisotropic object 53 is not caused as a premise. It should be noted that the fiber direction 56 of the anisotropic object 53 is a direction in which a fibrous structure that constitutes the anisotropic object 53 extends, for example. In this embodiment, the fiber direction of the anisotropic object 53 corresponds to an orientation direction of the optical anisotropic object.

It is assumed that an electric field vector of the incident light (the polarization light 3) of the first polarization direction 29 is I sin (ωt). Where I is an amplitude of the incident light, ω is an angular frequency of the incident light, and t is a time. Assuming that the angle between the fast axis 54 and the first polarization direction is φ, the electric fields of a slow axis component f and a fast axis component s when those exit from the anisotropic object 53 are respectively expressed by the following equation.

f=I sin(ωt)cos((φ)

s=I sin(ωt−δ)sin((φ)

It should be noted that δ is a phase difference between the fast axis component f and the slow axis component s.

The slow axis component f and the fast axis component s are incident on the second polarization element 31. In other words, the second polarization element 31 extracts the polarization component 5b of the second polarization direction from the slow axis component f and the fast axis component s. The electric field vector extracted by the second polarization element 31 is expressed by the following equation.

f*sin(φ)−s*cos(φ)=I cos(φ)sin(φ){sin(ωt)−sin(ωt−δ)}=I sin(2φ)sin(δ/2)cos(ωt−δ/2)

The intensity (the first intensity) of the electric field vector extracted by the second polarization element 31 is expressed by the square of I sin(2φ)sin(δ/2) which is the amplitude. In other words, the first intensity detected in a case of carrying out crossed nicols observation of the anisotropic object 53 is as follows.

I² sin²(2φ)sin²(δ/2)=I² sin²(2φ)sin²(π/λ)d|nₒ−nₑ|) (1)

Where λ is the wavelength of the incident light. In addition, d|nₒ|nₑ| t indicates an optical path difference between a normal light beam and an abnormal light beam and takes a value according to optical characteristics and the like of the anisotropic object 53. It should be noted that a similar result is obtained also in a case where the angle between the slow axis 55 and the first polarization direction is set to φ.

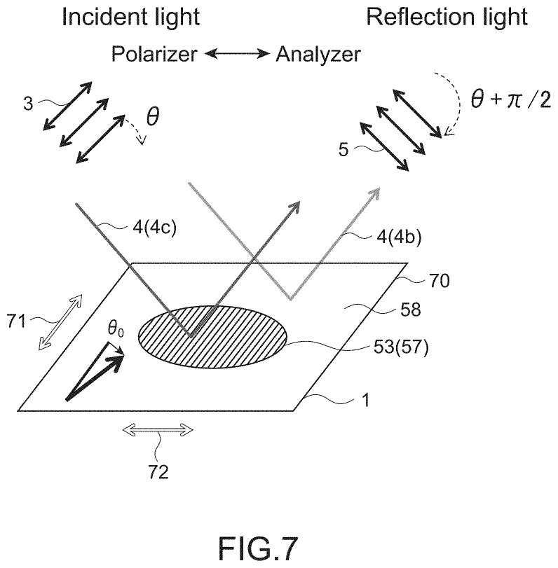

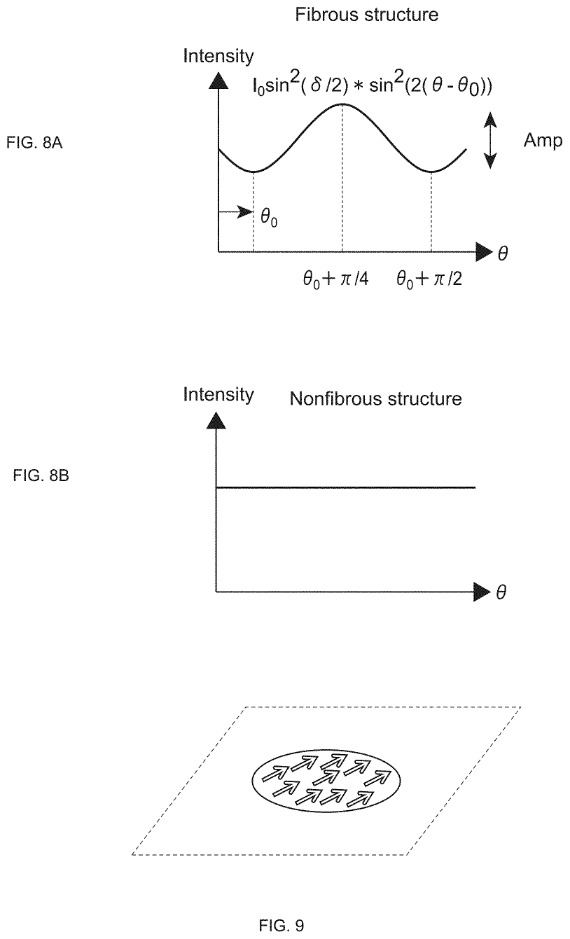

FIG. 6 is a graph showing the first intensity detected in a case of carrying out crossed nicols observation of the anisotropic object 53. The horizontal axis of the graph indicates the angle φ between the fast axis 54 of the anisotropic object 53 and the first polarization direction 29 and the vertical axis indicates the first intensity (the intensity of the polarization component 5b which is extracted by the second polarization element 31). The graph shown in FIG. 6 represents a change according to the angle φ of the first intensity expressed by Equation (1).

As indicated in Equation (1), the first intensity is a periodic function with a period of π/2 (90°) with respect to the angle φ. FIG. 6 shows a graph for two cycles from φ=0 to π (180°).

For example, in a case where the angle φ is 0, the intensity of the polarization component 5b is zero. In other words, in a case where the first polarization direction is orthogonal to the fiber direction 56 of the anisotropic object 53 (the direction of the slow axis 55), the first intensity reflected by the anisotropic object 53 and extracted by the second polarization element 31 is minimum.

Similarly, also in a case where the angle φ is π/2, i.e., in a case where the first polarization direction is parallel to the fiber direction 56 of the anisotropic object 53, the first intensity is minimum. It should be noted that sometimes the minimum value of the first intensity is not zero because a certain type and the like of the anisotropic object 53 to be observed causes random polarization due to its internal multiple reflection. In this case, for example, the graph shown in FIG. 6 is shifted upward.

On the other hand, in a case where φ is π/4, the intensity of the polarization component 5b is I² sin²(δ/2), maximum. In other words, in a case where the angle between the first polarization direction 29 and the fiber direction 56 of the anisotropic object 53 is π/4, the first intensity is maximum. As described above, when the angle of the first polarization direction 29 with respect to the fiber direction 56 of the anisotropic object 53 changes, the first intensity changes by an amplitude of I² sin²(δ/2) (a difference between the maximum value and the minimum value).

In actual measurement, the first intensity can sometimes change in accordance with the degree of sameness between the fiber directions 56 of the anisotropic object 53, i.e., an orientation which is the degree of orientation of the anisotropic object 53. For example, in a case where the fiber directions of the anisotropic object 53 are various, there is a possibility that the amplitude of the first intensity is lower in comparison with the case where the fiber directions of the anisotropic object 53 are the same.

Hereinafter, the amplitude of the first intensity will be referred to as Amp=I₀ sin²(δ/2). Io is a value depending on the orientation of the anisotropic object 53. In addition, as described above, δ is a phase difference between the fast axis component f and the slow axis component s caused by the anisotropic object 53 and is a value depending on the optical anisotropy of the anisotropic object 53.

FIG. 7 is a schematic view showing an example of crossed nicols observation. FIG. 8 is a diagram showing an example of a result of observation of crossed nicols observation.

FIG. 7 schematically shows an imaging range 70 by the image sensor 32, upper and lower directions 71 of the imaging range 70, and the left and right directions 72 orthogonal to the upper and lower directions 71. The imaging range 70 includes a fibrous structure 57 that is the anisotropic object 53 and a non-fibrous structure 58. The fibrous structure 57 is a structure in which double refraction of one axis direction is caused in the fiber direction 56. The non-fibrous structure 58 is a structure in which double refraction is not caused or a structure which has little orientation and in which double refraction is extremely small.

In addition, FIG. 7 schematically shows the polarization light 3 of the first polarization direction 29 which is incident on the imaging range 70 and the polarization component 5 of the second polarization direction 37 among beams of the reflection light 4 from the imaging range 70, which is extracted by the second polarization element 31. It should be noted that the illustration of the illumination system 20 and the imaging system 30 is omitted from FIG. 7.

As shown in FIG. 7, the polarization light 3 of the first polarization direction 29 is incident on the observation target 1 at an incident polarization angle θ. Here, the incident polarization angle θ is an angle of the polarization direction of the linearly polarized light with respect to the observation target 1 in a case where the linearly polarized light is incident on the observation target 1. Hereinafter, it is assumed that a state in which the upper and lower directions 71 of the imaging range 70 and the first polarization direction 29 are parallel is a state in which the incident polarization angle θ is zero. It should be noted that the method of setting the incident polarization angle θ is not limited and the incident polarization angle θ may be set by using the left and right directions 72 of the imaging range 70 as a reference, for example.

In crossed nicols observation, the intersection angle between the first polarization direction 29 and the second polarization direction 37 is maintained at substantially 90°. Therefore, the angle of the second polarization direction 37 with respect to the observation target 1 is θ+90° (θ+π/2). In this manner, the angles of the first polarization direction 29 and the second polarization direction 37 with respect to the observation target 1 are respectively expressed by using the incident polarization angle θ.

As described above, in this embodiment, the rotation control section 41 rotates the first polarization direction 29 and the second polarization direction 37 and the incident polarization angle θ changes. This rotation operation is performed so as to increase the incident polarization angle θ by a predetermined angle step, for example. The image sensor 32 performs imaging of the observation target 1 at each incident polarization angle θ and generates each image signal of the observation target 1 at each incident polarization angle θ.

In crossed nicols observation, the polarization component 5 of the second polarization direction 37 among beams of the reflection light 4 reflected by the observation target 1 is incident on the image sensor 32. The intensity of the polarization component 5 which is incident on this image sensor 32 is detected as the first intensity.

As shown in FIG. 7, the reflection light 4 reflected by the observation target 1 includes the reflection light 4c reflected by the anisotropic object 53 and the reflection light 4b reflected by the non-fibrous structure 58. The polarization component 5 of the second polarization direction among beams of the reflection light 4c and 4b is incident on the image sensor 32. It should be noted that reflection light reflected by the surface of the observation target 1 is omitted from FIG. 7.

By generating an image signal for each incident polarization angle θ as described above, it is possible to examine how the luminance and the like at each position in the imaging range 70 have changed along with a change in incident polarization angle θ. As a result, the change in first intensity according to the rotation operation can be analyzed in detail for each position of the observation target 1.

The graph of FIG. 8A is a graph showing an example of the first intensity detected in crossed nicols observation. FIG. 8A shows the intensity (the first intensity) of the polarization component 5 of the second polarization direction 37 which has been reflected by the anisotropic object 53. The horizontal axis of the graph indicates the incident polarization angle θ and the vertical axis indicates the intensity of the polarization component 5.

In a case of carrying out crossed nicols observation of the anisotropic object 53, the first intensity is expressed as the periodic function with respect to the angle φ as described in Equation (1). This angle φ can be expressed by using the incident polarization angle θ and a phase component θ₀. The relation between the incident polarization angle θ and the first intensity is expressed as follows.

I₀ sin²(δ/2)×sin² (2(θ−θ₀)) (2)

As described in Equation (2), the first intensity is the periodic function that fluctuates with a cycle of 90° with respect to the incident polarization angle θ. It should be noted that in the graph of FIG. 8A, the first intensity includes an offset due to randomization or the like of the polarization direction due to multiple reflection caused inside the observation target 1.

As shown in FIG. 8A, the first intensity is minimum at θ₀. In addition, the first intensity is maximum at θ₀+π/4 and is minimum again at θ₀+π/2. As described above, the incident polarization angle θ is increased from 0°, and the initial value with which the first intensity is minimum is the phase component θ₀.

In a case where the first polarization direction 29 and the fiber direction 56 of the anisotropic object 53 are parallel or orthogonal, the first intensity is minimum. Therefore, the phase component θ₀ indicates the direction orthogonal or parallel to the fiber direction 56 of the anisotropic object 53. In this manner, the information regarding the phase component θ₀ is information regarding the fiber direction 56 (the orientation direction) of the anisotropic object 53.

In addition, the amplitude of the first intensity Amp is I₀ sin²(δ/2). This amplitude Amp is expressed by the value (I₀) according to the orientation of the anisotropic object 53 and the value (δ) according to the optical anisotropy of the anisotropic object 53. In this manner, the information regarding the amplitude Amp is information regarding the orientation of the anisotropic object 53 and the anisotropy.

The graph of FIG. 8B is a graph showing another example of the first intensity detected in crossed nicols observation. The reflection light 4b reflected by the non-fibrous structure 58 does not have a particular polarization direction and the polarization direction is randomized. Therefore, the reflection light 4b includes a substantially constant proportion of the polarization component 5 of the second polarization direction irrespective of the value of the incident polarization angle θ.

As shown in the graph of FIG. 8B, substantially constant first intensity is detected irrespective of the incident polarization angle θ in a case of carrying out crossed nicols observation of the non-fibrous structure 58. Therefore, with the non-fibrous structure 58, the periodic change in first intensity as shown in FIG. 8A is not detected. It should be noted that a change in first intensity is substantially zero in a case where a structure in which double refraction is not caused or a region or the like having large specular reflection, which is covered with body fluid, is observed.

As described above, in a case where the first intensity changes with a cycle of π/2 with respect to the incident polarization angle θ, it is highly likely that the anisotropic object 53 is being observed. In contrast, in other cases, it is highly likely that the non-fibrous structure 58 is being observed. Therefore, it is possible to calculate identification information for identifying whether or not the observation target 1 includes the anisotropic object 53 by analyzing the change in first intensity according to the rotation operation.

FIG. 9 is a schematic view showing an example of a result of observation of crossed nicols observation. FIG. 9 shows an outer frame of an image constituted by an image signal generated in crossed nicols observation as the dotted lines.

In crossed nicols observation, regarding each position of the imaging range 70, a change in intensity of the polarization component 5 of the second polarization direction 37 according to the rotation operation is detected. On the basis of this detection result, for example, it is possible to display the region included in the anisotropic object 53 in an emphasis state or to display the fiber directions 56 of the anisotropic object 53 with the arrows as shown in FIG. 9. As a matter of course, a process such as mapping information regarding the orientation of the anisotropic object 53 and the anisotropy or the like may be performed.

Hereinafter, observation of the observation target 1 will be described specifically.

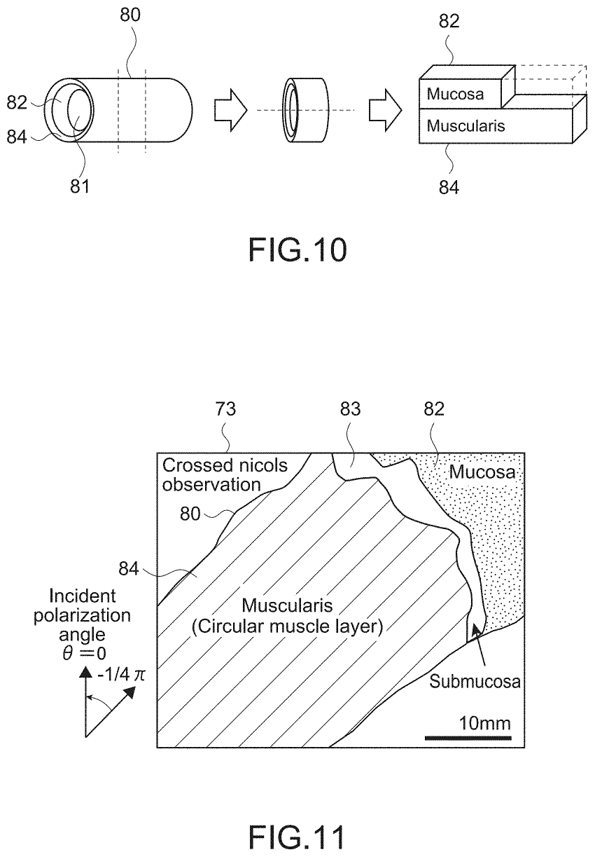

FIG. 10 is a schematic view for describing the observation target 1. FIG. 11 is a schematic view showing an example of an image of the observation target 1 imaged in crossed nicols observation. Hereinafter, the description will be given by showing a rectum of a pig as an example of the observation target 1.

FIG. 10 schematically shows a rectum 80 of a pig. The rectum 80 is a tubular structure and has a lumen 81. Digested food and the like pass through the lumen 81. The rectum 80 includes a mucosa 82, a submucosa 83, and a muscle layer 84 (muscularis) from the inside (from the lumen 81 side). FIG. 10 schematically shows the mucosa 82 and the muscle layer 84 that constitute the rectum 80. It should be noted that the illustration of the submucosa 83 is omitted.

The inside of the muscle layer 84 is constituted by a circular muscle layer and the outside of the circular muscle layer is constituted by a longitudinal muscle layer. Muscle fibers that constitute the circular muscle layer are oriented in a direction substantially orthogonal to a direction in which the rectum 80 extends. In other words, a muscle fiber direction of the circular muscle layer is a direction along the inner periphery surrounding the lumen 81. In addition, muscle fibers that constitute the longitudinal muscle layer are oriented in a direction substantially parallel to the direction in which the rectum 80 extends.

As shown in FIG. 10, a part of a tubular structure is cut out by cutting the rectum 80. The mucosa 82 inside the rectum 80 is exposed by incising the cut-out rectum 80. Then, the muscle layer 84 is exposed by pealing off a part of the exposed mucosa 82. FIG. 10 schematically shows the pealed-off mucosa 82 as the dotted lines. At this time, the circular muscle layer can be seen through the exposed portion of the muscle layer 84. This mucosa 82 and the site at which the circular muscle layer (the muscle layer 84) is exposed are used as the observation target 1. Hereinafter, the exposed circular muscle layer will be simply referred to as the muscle layer 84.

FIG. 11 schematically shows an observation image 73 of the pig's rectum 80 (the observation target 1) imaged in crossed nicols observation. The observation image 73 includes the exposed muscle layer 84 (the circular muscle layer) and the mucosa 82. In addition, the submucosa 83 is present at the boundary between the muscle layer 84 and the mucosa 82. It should be noted that FIG. 11 schematically shows the muscle fiber direction of the muscle layer 84 as the oblique lines and the mucosa 82 as the dots. No oblique lines, dots, and the like are displayed in the actual observation image 73.

As the observation image 73, the rectum 80 is imaged such that the muscle fiber direction 56 of the exposed muscle layer 84 is a direction extending from the lower left to the upper right of the observation image 73. More specifically, the muscle fiber direction 56 is set to cross the upper and lower directions 71 of the observation image 73 at an angle of substantially π/4.

The upper and lower directions 71 of the observation image 73 are a direction similar to the upper and lower directions 71 of the imaging range 70 shown in FIG. 7. Therefore, for example, in a case where the incident polarization angle θ is π/4, the first polarization direction 29 and the muscle fiber direction of the muscle layer 84 are substantially parallel to each other. It corresponds to the state in which the phase component θ₀ shown in the graph of FIG. 8A is substantially π/4. In other words, the intensity of the polarization component 5 of the second polarization direction 37 is minimum where θ=π/4.

In addition, for example, the state in which the incident polarization angle θ is 0 corresponds to the state of θ₀−π/4. As described above, the intensity of the polarization component 5 of the second polarization direction 37 is a periodic function that fluctuates with a cycle of π/2 and is a maximum value at an angle of the phase component θ₀±π/4. Therefore, in the state of the incident polarization angle θ=0, the intensity of the polarization component 5 similar to the maximum value in θ₀+45° shown in the graph of FIG. 8A is detected. Hereinafter, imaging of the observation target 1 (the rectum 80) is performed in the arrangement shown in FIG. 11.

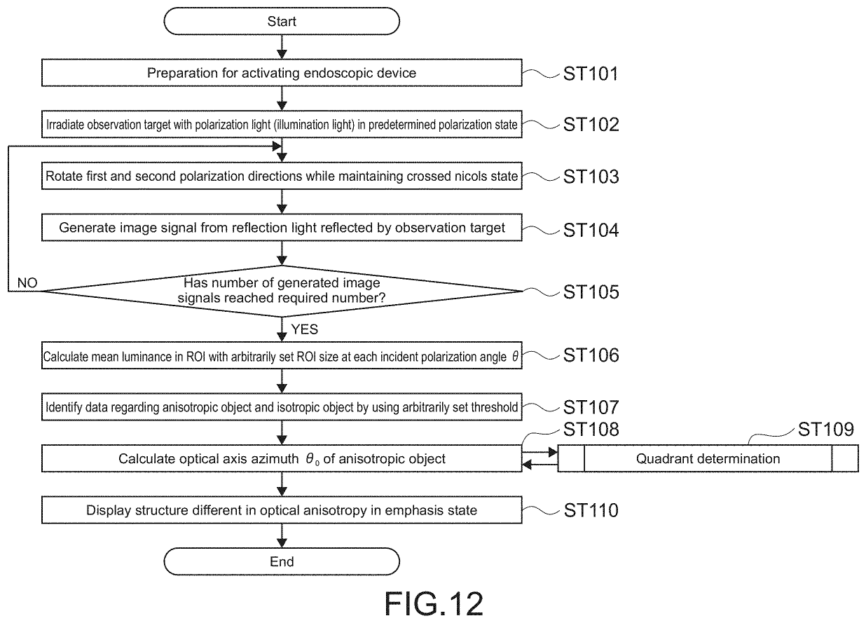

FIG. 12 is a flowchart showing an example of observation of a biological tissue. As shown in FIG. 12, preparation for activating the endoscopic device 100 is first performed (Step 101). For example, respective sections such as the light source 21, the image sensor 32, and the controller 40 are activated. In addition, an operator such as a doctor inputs various kinds of parameters for observation using the endoscopic device 100 (such as amounts of light of the light source 21 and sensitivity of the image sensor 32) to the controller 40 or the like.

Polarization light in a predetermined polarization state is generated from the illumination light 2, and the polarization light is emitted to the observation target 1 (Step 102). In other words, the first polarization element 22 generates the polarization light 3 of the first polarization direction 29, and the polarization light 3 is emitted to the observation target 1.

The first polarization direction 29 is set such that the incident polarization angle θ is 0. In other words, the first polarization direction 29 and the upper and lower directions 71 of the imaging range 70 (the observation image 73) of the image sensor 32 are set to be parallel to each other. At this time, the second polarization direction 37 is set in such a manner that the substantially crossed nicols relation is established between the first polarization direction 29 and the second polarization direction 37.

The rotation control section 41 rotates the first polarization direction 29 and the second polarization direction 37 while maintaining the substantially crossed nicols state (Step 103). In this embodiment, each of the polarization directions is rotated by an angle step θs that has been set in advance. Details of the angle step θs will be described later.

In addition, the rotation may be omitted in a case where the process in Step 103 is performed for the first time after the preparation for activation is performed in Step 101. In other words, when the process in Step 103 is performed for the first time, rotation is made by an angle step θs=0°. When the process in Step 103 is performed for the second or subsequent times, the first polarization direction 29 and the second polarization direction 37 are rotated by an angle step θs that has been set in advance.

On the basis of the reflection light 4 reflected by the observation target 1, the image sensor 32 generates an image signal of the observation target 1 (Step 104). In other words, the image signal is generated on the basis of the polarization component 5 of the second polarization direction that has been extracted by the second polarization element 31 among beams of the reflection light 4 reflected by the observation target 1. In this embodiment, it is possible to generate the image signal capable of configuring a color image of the observation target 1. As a matter of course, it is also possible to generate an image signal capable of configuring a black and white image or the like. The generated image signal is output to the intensity detection section 42.

It is determined whether or not the number of generated image signals has reached a required number (Step 105). In a case where it is determined that the number of image signals has not reached the required number (No in Step 105), the process returns to Step 103 and a loop process is performed.

The angle step θs at Step 103 and the required number N at Step 105 will be described. As described above, the polarization light 3 of the first polarization direction is linearly polarized light. Therefore, a state in which the first polarization direction 29 is rotated by π (180°) can be considered as a polarization state similar to the state before rotation. Therefore, for example, a state in which the first polarization direction is rotated by the angle α is a state similar to the state in which it is rotated by π+α.

In this embodiment, the first polarization direction is rotated such that the incident polarization angle θ takes a value from 0 to π. Accordingly, for example, additional imaging is not required and the time and the like required for observation can be shortened.

The required number N at Step 105 is the number of times of imaging performed by changing the incident polarization angle θ into an angle in a range from 0 to π. The required number N is set as appropriate such that observation can be performed with desired accuracy, for example. In addition, the angle step θs at Step 103 is set such that θs=π/(N−1) is established.

In this embodiment, the required number N is set to 17 and the angle step θs is set to π/16 (=11.25°). In other words, at Step 103, the first polarization direction 29 and the second polarization direction 37 are rotated such that the incident polarization angle θ is 0, π/16, . . . , π. Accordingly, the change and the like in the polarization component 5 along with the rotation operation can be detected with a sufficient accuracy.

It should be noted that the method or the like of setting the required number N and the angle step θs is not limited and may be set as appropriate in accordance with observation accuracy and the like. In addition, as described above, it is not limited to the case of changing the incident polarization angle θ in the range of 0 to π. For example, the range or the like in which the incident polarization angle θ is changed so as to obtain desired observation accuracy may be set as appropriate.

In a case where it is determined that the required number N of image signals have been obtained (Yes of Step 105), a process of calculating the biological tissue information of the observation target 1 on the basis of the N image signals is started.

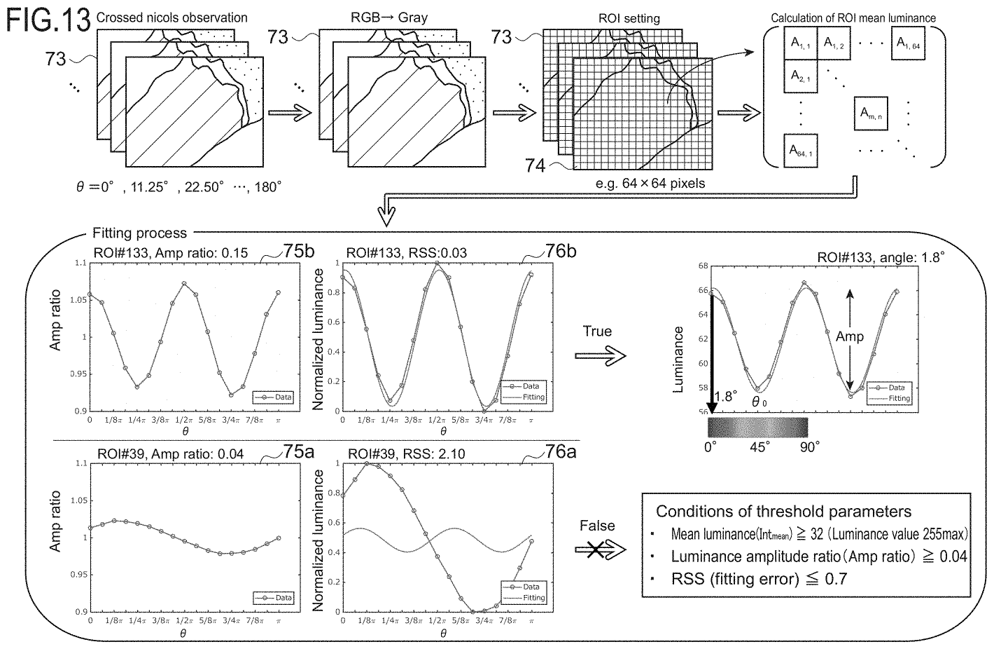

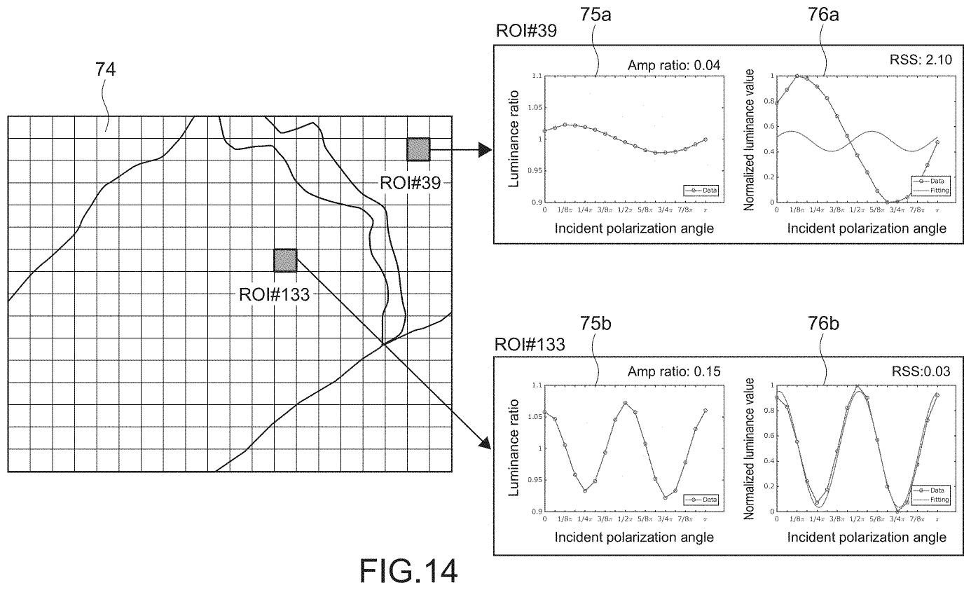

FIG. 13 is a diagram for describing an example of the process of calculating the biological tissue information on the basis of the image signal generated in crossed nicols observation. FIG. 14 is a diagram showing a specific example of the process of calculating the biological tissue information shown in FIG. 13.

FIG. 13 sequentially shows respective processes for calculating the biological tissue information on the basis of the image signal. The image signal generated by the image sensor 32 is output to the intensity detection section 42. The intensity detection section 42 detects the first intensity for each pixel of the image signal. Here, a process of converting the RGB value of each pixel into the gray scale is performed and a luminance value indicated by the gradation of gray scale is detected as the first intensity. The detected first intensity (the image signal converted into the gray scale) is output to the analysis section 43.

The analysis section 43 sets a plurality of analysis regions (ROIs), into which the observation image 73 constituted by the image signal is to be divided, and calculates biological tissue information with respect to each of the plurality of analysis regions. In this embodiment, the analysis regions correspond to target regions. Hereinafter, the analysis region will be referred to as an ROI 74.

First of all, the analysis section 43 sets the ROI 74 having a predetermined size with respect to each image signal converted into the gray scale and calculates a mean luminance in the ROI 74 (Step 106).

The size of the ROI 74 can be set as appropriate in accordance with the resolution and the like for observing the observation target 1, for example. In this embodiment, the ROI 74 of 64 pixels×64 pixels is used. With this ROI 74, the observation image 73 of 1280 pixels×1024 pixels can be divided into blocks of 20×16, for example. As a matter of course, not limited thereto, the ROI 74 having a desired size may be set as appropriate.

The analysis section 43 calculates the average value (the mean luminance) of the first intensity of the pixels included in the ROI 74 for each set ROI 74. FIG. 13 schematically shows pixels included in one ROI 74 and its luminance value A_{m,n}. It should be noted that m and n are integers from 1 to 64 and are indicators representing the position of each pixel within the ROI. An average value of the first intensity is calculated by dividing the sum in the ROI 74 of this luminance value A_{m,n} by the number of pixels (64×64) in the ROI 74.

The process of calculating the average value of the first intensity of the ROI 74 is performed on each of N image signals (the observation image 73). Therefore, an average value of the first intensity in a case where the incident polarization angle θ is 0, π/16, . . . , π is calculated for each ROI. As described above, data regarding the average value of the first intensity according to the incident polarization angle θ calculated for each ROI is used as first intensity data related to a change in first intensity according to the rotation operation.

FIG. 14 shows graphs 75a and 75b of the first intensity data calculated at the ROI #39 and the ROI #133. The horizontal axis of the graph 75a or 75b indicates the incident polarization angle θ and the vertical axis indicates a luminance ratio. Here, the luminance ratio is a value obtained by dividing the data point (the average value of the first intensity) calculated for one ROI 74 by an average value (I_{average}) of N data points.

As described above, the amplitude of the first intensity data in each ROI 74 can be easily compared by representing the first intensity data by the use of the luminance ratio. In this embodiment, the luminance amplitude ratio (Amp ratio) is calculated as the amplitude of the first intensity data. The luminance amplitude ratio is a value obtained by dividing the difference (the amplitude) between the maximum value and the minimum value of the N data points by an average value I_{average} of the N data points. In other words, the luminance amplitude ratio corresponds to the amplitude of the luminance ratio in the graphs 75a and 75b.

As shown in FIG. 14, in the ROI #39, the luminance ratio does not greatly change even when the incident polarization angle θ changes. It can be seen that the mucosa 82 exists at the position at which the ROI #39 is set. The luminance amplitude ratio calculated in the ROI #39 is 0.04.

It should be noted that the graph 75a of the ROI #39 shows small fluctuations with a π (180°) cycle. It can be considered that such a phenomenon is caused by various factors such as leakage of part of specular reflection light in a case where the extinction ratio of the polarizer is not sufficiently large, stray light due to reflection or the like outside the imaging range 70, and other leaking light, for example.

The luminance ratio in the ROI #133 is a periodic function that fluctuates with a cycle of π/2 (90°) with respect to the incident polarization angle θ. Therefore, it can be seen that the muscle layer 84 exists at the position at which the ROI #133 is set. In addition, the luminance amplitude ratio which is the amplitude of the graph 75b is 0.15 and takes a sufficiently large value in comparison with the ROI #39 on the mucosa 82.

The analysis unit performs a fitting process using a predetermined function with respect to the first intensity data. FIG. 14 shows graphs 76a and 76b which are results of the fitting process on the first intensity data calculated for the ROI #39 and the ROI #133. The horizontal axis of the graph 76a or 76b indicates the incident polarization angle θ and the vertical axis indicates a luminance value normalized at the maximum value.

In this embodiment, a predetermined function f(θ)=A×sin²(2(θ−B))+C is set by using the function described in Equation (2) as a reference. Parameters A and B are parameters representing amplitude information and phase information of the predetermined function f(θ). Therefore, it can also be said that the parameters A and B are parameters corresponding to the amplitude Amp and the phase component θ₀ of Equation (2). In this embodiment, the predetermined function f(θ) corresponds to a predetermined periodic function. It should be noted that the parameter C is a parameter representing an amount of offset of the predetermined function f(θ).

In the fitting process, such parameters A and B that the predetermined function f(θ) fits the first intensity data are calculated. In addition, a residual sum of squares (RSS) is calculated as a parameter for assessing the discordance between the predetermined function f(θ) and the first intensity data. It should be noted that a specific method and the like for the fitting process are not limited, and a process using a least squares method or the like, for example, may be performed as appropriate.

As shown in FIG. 14, the predetermined function f(θ) is hardly fitted to the first intensity data calculated for the ROI #39. As a result of the fitting process for the ROI #39, the calculated residual sum of squares is 2.10.

On the other hand, such parameters A and B that the predetermined function f(θ) can be sufficiently fitted to the first intensity data calculated for the ROI #133 are calculated. The result of the fitting process for the ROI #133, the residual sum of squares is 0.03. It means that the ROI #133 is in accord with the predetermined function f(θ) more sufficiently than the ROI #39.

Therefore, the amplitude Amp and the phase component θ₀ of the periodic function expressed by Equation (2) can be calculated by calculating the parameters A and B in the ROI 74 including the anisotropic object 53 (the muscle layer 84). As shown in described in the graph of FIG. 8A, the information regarding the phase component θ₀ is the information regarding the orientation direction of the anisotropic object 53. In addition, information regarding the amplitude Amp is information regarding the orientation of the anisotropic object 53 and the anisotropy.

As described above, the analysis section 43 performs the fitting process using the predetermined function f(θ), calculates the information regarding the phase component θ₀ on the basis of phase information B of the predetermined function f(θ) which is obtained as a process result of the fitting process, and calculates the information regarding the amplitude Amp on the basis of amplitude information A of the predetermined function f(θ).

In addition, the information regarding the phase component θ₀ and the information regarding the amplitude Amp, which have been calculated, are stored as the biological tissue information after an identification process of the anisotropic object 53 to be described later. In this embodiment, the information regarding the phase component θ₀ corresponds to first information regarding an orientation direction of the optical anisotropic object. In addition, the information regarding the amplitude Amp corresponds to the second information regarding the orientation of the optical anisotropic object and the anisotropy.

When the fitting process ends, a process of identifying signals of the anisotropic object 53 (the fibrous structure 57) and an isotropic object (the non-fibrous structure 58) using threshold parameters is performed (Step 107). FIG. 13 shows conditions of the threshold parameters. In this embodiment, the mean luminance (Intₘₑₐₙ), the luminance amplitude ratio (Amp ratio), and the residual sum of squares (RSS) of the ROI 74 are used as the threshold parameters. It should be noted that the threshold parameters can be changed as appropriate in a manner that depends on an imaging condition, an object to be imaged, and the like.

The average value (I_{average}) of the N data points, for example, is used as the mean luminance Intₘₑₐₙ. The mean luminance Intₘₑₐₙ is a parameter indicating the brightness of the ROI 74. Therefore, the observation target 1 and the background of the observation target 1 can be identified by comparing the mean luminance Intₘₑₐₙ with a predetermined threshold. As shown in FIG. 13, in a case where the luminance value (the first intensity) is expressed by an 8-bit scale 256 gradation, Intₘₑₐₙ≥32 is set for the condition related to the mean luminance to thereby exclude dark portions in the image from analysis. The calculation speed is thus increased.

The luminance amplitude ratio is a parameter indicating the levels of the orientation and the anisotropy. For example, in a case where the luminance amplitude ratio is small, it is highly likely that the orientation and the anisotropy are small and a site which is not the anisotropic object 53 is being observed. On the contrary, in a case where the luminance amplitude ratio is large, it is highly likely that the anisotropic object 53 is being observed. The condition related to the luminance amplitude ratio is set to Amp ratio 0.04.

The residual sum of squares is a parameter indicating the degree of accordance between the first intensity data and the predetermined function f(θ) as described above. In other words, it can be said that as the residual sum of squares becomes smaller, a fitting error of sin²(2θ) is smaller. In this case, it is highly likely that the first intensity data is a periodic function that fluctuates with a cycle of π/2 with respect to the incident polarization angle θ. The condition related to the residual sum of squares is set to RSS≤0.7.

The analysis section 43 identifies whether or not each ROI includes the anisotropic object 53 on the basis of the above-mentioned condition. For example, the ROI #133 is determined to satisfy (True) the conditions of the threshold parameters. Therefore, the ROI #133 is identified to include the anisotropic object 53. In addition, for example, the ROI #39 is determined not to satisfy (False) the conditions of the threshold parameters. Therefore, the ROI #39 is identified not to include the anisotropic object 53. Here, the threshold parameters are predicted to be different from an optimum value in a manner that depends on a measurement target, an illumination condition, and the like. Therefore, it is necessary to revise the parameters for correct identification as appropriate.

On the basis of the identification result, the analysis section 43 calculates identification information for identifying whether or not the observation target 1 includes the anisotropic object 53 as the biological tissue information of the observation target 1. In other words, information indicating whether or not each ROI includes the anisotropic object 53 is calculated as the identification information.

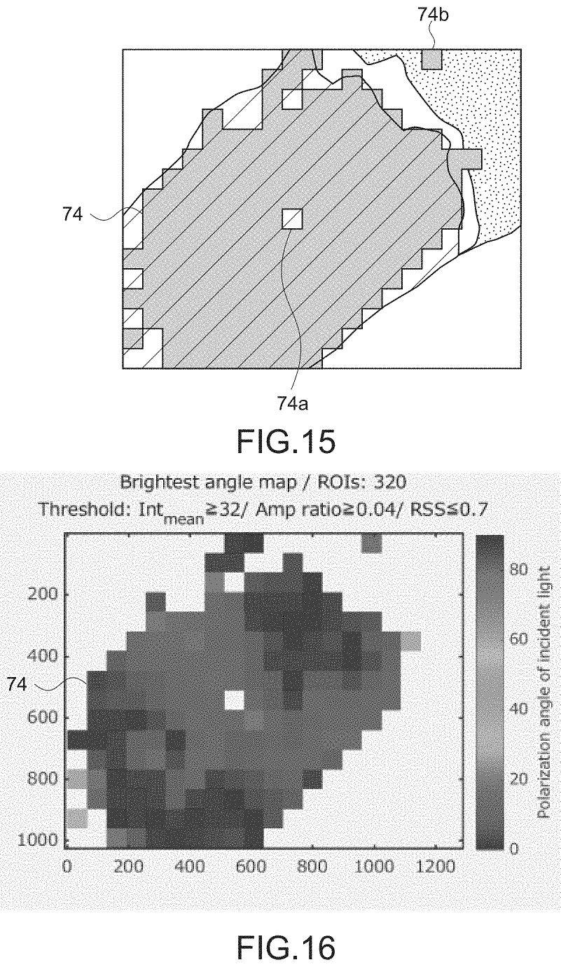

FIG. 15 is a schematic view showing an example of the identification result of the anisotropic object 53 according to crossed nicols observation. The ROI 74 identified to include the anisotropic object 53 is shown as a gray region. As shown in FIG. 15, at the site at which the muscle layer 84 is exposed, most ROIs 74 are identified to include the anisotropic object 53. On the other hand, at the site at which the mucosa 82 or the submucosa 83 is exposed, most ROIs 74 are identified not to include the anisotropic object 53.

As described above, by calculating the identification information, the muscle layer 84 including the anisotropic object 53 and other sites can be identified with high accuracy. In addition, a ROI 74a identified not to include the anisotropic object 53 on the muscle layer 84, a ROI 74b identified to include the anisotropic object 53 on the mucosa 82, and the like are calculated in the example shown in FIG. 15. In a case where such an identification result is available, for example, it is also possible to detect a local abnormality or the like in the muscle layer 84 or the mucosa 82.

When the identification process ends, the process result of the fitting process of the ROI 74 identified to include the anisotropic object 53 is stored as the biological tissue information. For example, as shown in FIG. 13, regarding the ROI #133, the phase component θ₀ related to the first intensity data, the amplitude Amp, and the like are stored. Also regarding other ROIs 74, a similar process is performed and is stored as the biological tissue information of the observation target 1.

The stored biological tissue information includes information regarding the muscle fiber direction of the muscle layer 84 in each ROI 74, the orientation and the anisotropy of muscle fibers, and the like. Besides, the type of data and the like stored as the biological tissue information are not limited. Desired information regarding the anisotropic object 53 can be mapped by using the biological tissue information.

FIG. 16 is a schematic view showing an example of the biological tissue information calculated in crossed nicols observation. FIG. 16 shows a result of mapping the incident polarization angle θ at which the luminance value of each ROI 74 has a peak as an example of the biological tissue information. Here, the incident polarization angle θ is indicated by using a color map corresponding to the angle of 0° to 90°. Accordingly, the incident polarization angle θ at which the luminance value has a peak can be easily observed.

For example, as shown in FIG. 13, the incident polarization angle θ=1.8° is a peak of the luminance value in the ROI #133. By using the fitting process as described above, a change in first intensity and the like can be expressed in more detail than the angle step θs. As a result, various characteristics about the anisotropic object 53 can be calculated with high accuracy and the observation target 1 can be observed in detail.

Referring back to FIG. 12, when the identification process ends and the biological tissue information is stored, a process of calculating the fiber direction 56 of the anisotropic object 53 is performed (Step 108). In this embodiment, in order to calculate the fiber direction 56 of the anisotropic object 53, a process of determining a quadrant including the fiber direction 56 is performed (Step 109). Details of this process of determining the quadrant will be described later.

When the fiber direction 56 of the anisotropic object 53 is calculated, a structure different in optical anisotropy is displayed in an emphasis state (Step 110). For example, on the basis of identification results (see FIG. 15) between the anisotropic object 53 and other structures, the analysis section 43 generates an emphasis image or the like in which the ROI 74 including the anisotropic object 53 is emphasized. The generated emphasis image is displayed on the display unit 50.

For example, as shown in FIG. 15, the image in which the ROI 74 identified to include the anisotropic object 53 is emphasized is included in the emphasis image according to this embodiment. In addition, for example, the fiber direction 56 that is the orientation direction of the anisotropic object 53, i.e., the phase component θ₀ may be displayed in different colors using a color map similar to the map of FIG. 16. In addition, not limited to the color map, an emphasis image in which the fiber direction 56 of each ROI 74 is indicated with an arrow or the like, for example, may be generated. Alternatively, the color map and the arrow may be used in combination.

In addition, for example, an image on which the orientation of the anisotropic object 53, the strength of the anisotropy, and the like have been mapped or the like may be generated. Besides, the image generated by the analysis section 43, the type of information displayed, and the like are not limited and desired parameters may be displayed as appropriate. Accordingly, the biological tissue which is the observation target 1 can be sufficiently observed in detail.

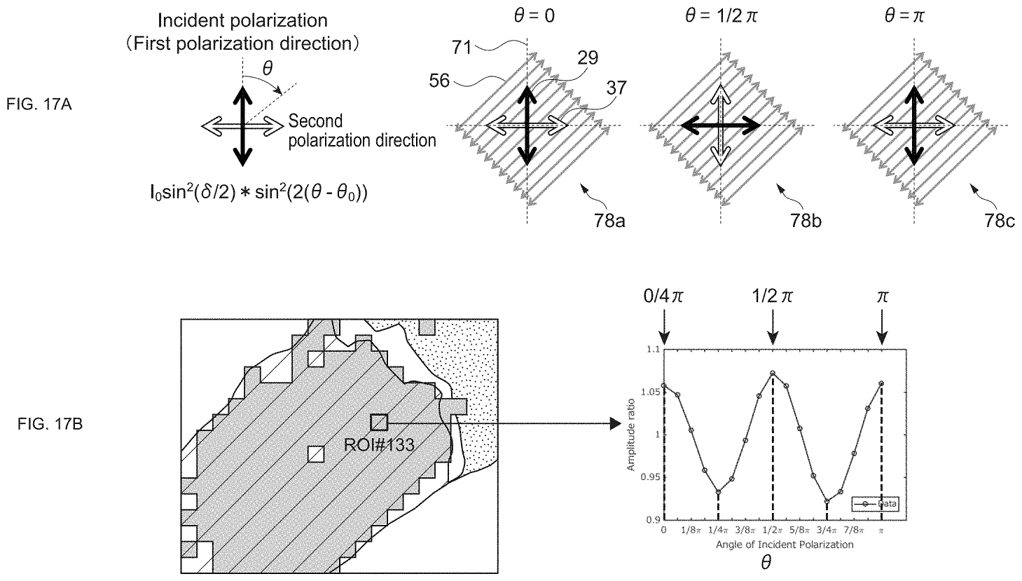

Hereinafter, the process of determining a quadrant at Step 109 will be described. In the process of determining a quadrant, the information regarding the fiber direction 56 (the orientation direction) of the anisotropic object 53 that has been stored as the biological tissue information is used. First of all, the information regarding the fiber direction calculated in crossed nicols observation will be described with reference to FIGS. 17 to 19.

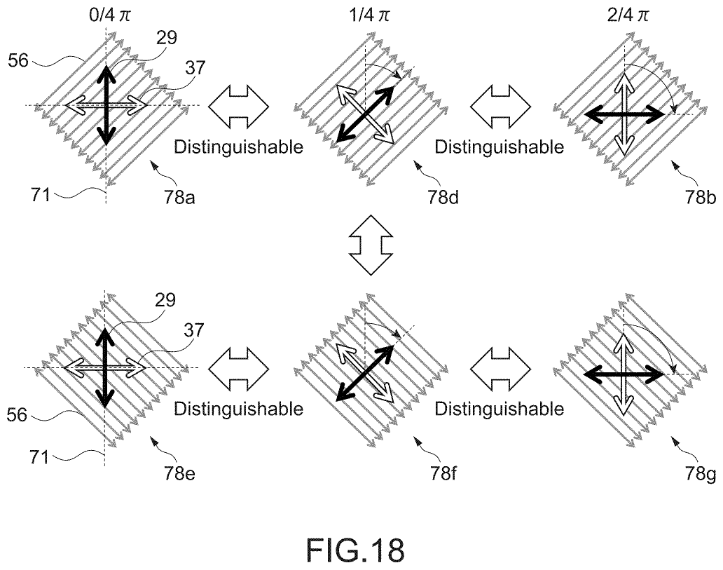

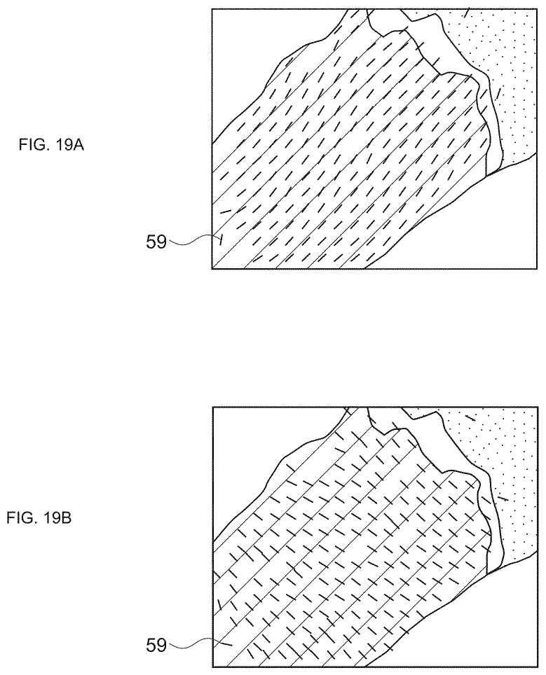

FIGS. 17 and 18 are diagrams for describing a relation between the incident polarization angle θ and the fiber directions in crossed nicols observation. FIG. 17A schematically shows the first polarization direction 29 and the second polarization direction 37 which are orthogonal to each other and the fiber direction 56 with arrows indicating the respective directions. It should be noted that the fiber direction 56 is set to extend from the lower left to the right upward. In addition, the angle between the upper and lower directions 71 and the fiber direction 56 is π/4 (45°).

As shown in FIG. 17A, the state 78a of the incident polarization angle θ=0 is a state in which the first polarization direction 29 and the upper and lower directions 71 are parallel. Therefore, in a case where θ=0 is established, the angle between the first polarization direction 29 and the fiber direction 56 is π/4. In this case, as described in Equation (2), the intensity (the first intensity) of reflection light reflected by the anisotropic object 53 which is detected in crossed nicols observation is maximum. Similarly, also in the states 78b and 78c in which θ=π/2 (90°) and θ=π(180°) are established, the first intensity is maximum. In other words, in a case where θ=k×π/2 (k: integer) is established, the first intensity in crossed nicols observation is maximum.

FIG. 17B shows a graph showing a change in first intensity with respect to the incident polarization angle θ at the ROI #133 shown in FIG. 14. At the position at which the ROI #133 is set, an angle between the fibers (muscle fibers) direction 56 of the muscle layer 84 which is the anisotropic object 53 and the upper and lower directions 71 is π/4 (45°). It is arrangement similar to that in the fiber direction 56 described in FIG. 17A. Therefore, as shown in the graph of FIG. 17B, in a case where the incident polarization angle θ is 0, π/2, and π, a peak value of the first intensity is detected.

The state 78a in which θ=0 is established, the state 78d in which θ=π/4 is established, and the state 78b in which θ=π/2 is established in a case where the angle between the fiber direction 56 and the upper and lower directions 71 is π/4 (45°) are shown on the upper side of FIG. 18. In addition, the state 78e in which θ=0 is established, the state 78f in which θ=π/4 is established, and the state 78g in which θ=π/2 is established in a case where the angle between the fiber direction 56 and the upper and lower directions 71 is ¾π (135°) are shown on the lower side of FIG. 18.

In a case where the angle between the fiber direction 56 and the upper and lower directions 71 is π/4, the first intensity takes a peak value in the state 78a or 78b in which the incident polarization angle θ is 0 or π/2. Further, the first intensity takes a bottom value in the state 78d in which the incident polarization angle θ is π/4 (see the graph of FIG. 17B). Similarly, also in a case where the angle between the fiber direction 56 and the upper and lower directions 71 is ¾π, the first intensity takes a peak value in the state 78e and 78g in which the incident polarization angle θ is 0 or π/2. In addition, the first intensity takes a bottom value in the state 78f in which the incident polarization angle θ is π/4.

As described above, also in a case where the fiber direction 56 is rotated by π/2 with respect to the upper and lower directions 71, the first intensity takes a peak value in θ=0 or π/2 and takes a bottom value π/4. In other words, in a case where the anisotropic objects 53 whose the fiber directions 56 are different from each other by π/2 are each observed in crossed nicols observation, a change in first intensity substantially similar to each other in the respective anisotropic objects 53 is detected.

It should be noted that irrespective of the angle between the fiber direction 56 and the upper and lower directions 71, the state 78a (78e) in which θ=0 is established and the state 78d (78f) in which θ=π/4 is established are distinguished as states different in value of the first intensity. Similarly, the state 78d (78f) in which θ=π/4 is established and the state 78b (78g) in which θ=π/2 is established are also distinguishable using the value of the first intensity.

Therefore, a difference of a relative angle of the fiber direction 56 for each ROI can be detected by comparing the incident polarization angle θ at which a peak value or a bottom value of a change in first intensity is detected for each ROI, for example. In other words, it can be said that a relative angle of the fiber direction 56 in the range of 0 to π/2 is detected in crossed nicols observation.

FIG. 19 is a schematic view showing an example in a case where the fiber direction 56 is displayed using information regarding the fiber direction 56 of the anisotropic object 53 calculated in crossed nicols observation. It should be noted that regarding each ROI determined to include the anisotropic object 53, the fiber direction 56 of the anisotropic object 53 in each ROI is shown with a straight line 59 in FIGS. 19A and 19B. In other words, the direction in which each straight line 59 extends corresponds to the fiber direction 56 in each ROI.

In FIG. 19A, the angle between the fiber direction 56 and the upper and lower directions 71 is expressed using the phase component θ₀. In other words, the direction parallel to the direction indicated by the phase component θ₀ is plotted as the fiber direction 56. It should be noted that the fiber direction 56 of the muscle layer 84 is arranged to be substantially 45° with respect to the upper and lower directions 71. Therefore, it can be said that the result shown in FIG. 19A is a result of suitably detecting the fiber direction 56 of the muscle layer 84.

In FIG. 19B, each fiber direction 56 is displayed such that the angle between the fiber direction 56 and the upper and lower directions 71 is the phase component θ₀+90°. As described with reference to FIG. 18 and the like, there is still a possibility that the fiber direction 56 as shown in FIG. 19B is detected in a case of using the calculated phase component θ₀ only in crossed nicols observation. It should be noted that in a case of a typical observation target, the fiber direction of the target object can be often assumed in accordance with the anatomical knowledge, and a sufficiently suitable fiber direction may be detected even with such a detection result.

In this embodiment, observation with one nicol of the observation target 1 (one nicol observation) is performed in addition to crossed nicols observation. Then, the quadrant determination with respect to the fiber direction 56 of the anisotropic object 53 is performed on the basis of the observation result of one nicol observation. It should be noted that in this embodiment, one nicol observation corresponds to observation performed in a state in which the second polarization element 31 has been removed from the optical path of the reflection light 4. In other words, one nicol observation corresponds to observation performed in the state in which the third polarization section has been configured.

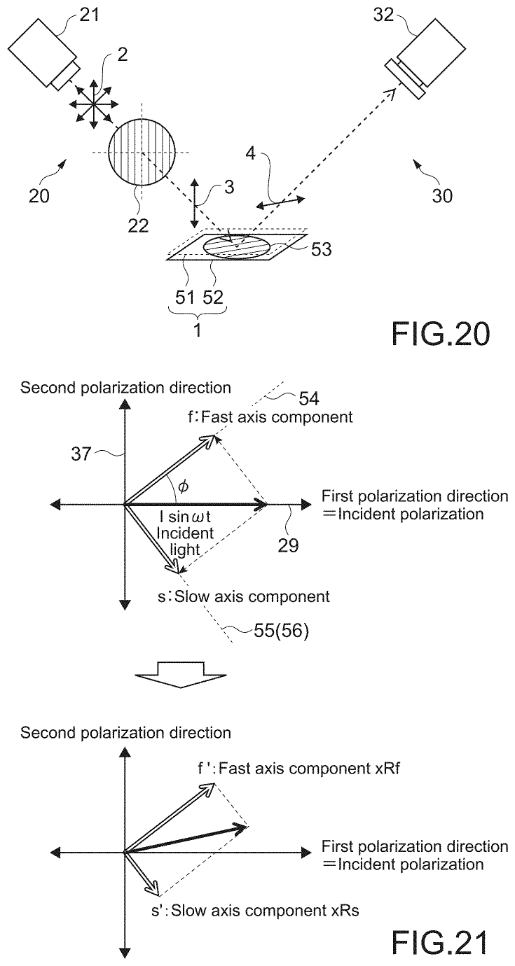

FIG. 20 is a schematic view showing an example of observation of the anisotropic object 53 according to one nicol observation. The configuration shown in FIG. 20 is a configuration obtained by removing the second polarization element 31 (the polarizing plate 36 having the second polarization axis 35) from the configuration for crossed nicols observation described in FIGS. 2 and 4 or the like. By constituting such an imaging system 30, the reflection light 4 reflected by the observation target 1 can be extracted while maintaining the polarization state in the imaging system 30. It should be noted that the first polarization element 22 is used in a manner similar to that in crossed nicols observation.

The polarization light 3 of the first polarization direction which has been emitted from the illumination system 20 is reflected by the observation target 1. This reflection light 4 is extracted with the polarization state maintained and is incident on the image sensor 32. Then, the image sensor 32 and the intensity detection section 42 detect the second intensity which is the intensity of the extracted reflection light 4. In other words, it can also be said that the second intensity is the intensity of the reflection light 4 detected by one nicol observation using the first polarization element 22.

FIG. 20 schematically shows the reflection light 4 reflected by the anisotropic object 53 in the inside 52 of the observation target 1. In actual observation, the reflection light 4 extracted while maintaining the polarization state includes specular reflection components on the observation target 1, components reflected by the non-fibrous structure 58, and the like. Therefore, the second intensity includes the intensity of the reflection light 4 and specular reflection intensity due to the anisotropic object 53 and the non-fibrous structure 58.

The inventor of the present technology has considered the second intensity detected in a case where the reflection light 4 of the anisotropic object 53 has been subjected to one nicol observation as follows. FIG. 21 is a schematic view for describing the consideration. Hereinafter, the description will be given assuming that the direction parallel to the slow axis 55 is the fiber direction of the anisotropic object 53. In addition, it is assumed that reflection coefficients of directions parallel to the respective axes of the fast axis 54 of the anisotropic object 53 and the slow axis 55 are Rf and Rs.

It is assumed that the electric field vector of the incident light (the polarization light 3) is I sin(ωt). As shown in the figure on the upper side of FIG. 21, the incident light can be decomposed into and represented by the fast axis component f and the slow axis component s. Assuming that the angle between the fast axis 54 and the first polarization direction is φ, electric field vectors of the fast axis component f′ and the slow axis component s′ reflected by the anisotropic object 53 are respectively expressed by the following equations.

f′=I sin(ωt)cos((φ)

s′=I sin(ωt−δ)sin((φ)

The intensity (the second intensity) of the electric field vector reflected by the anisotropic object 53 is expressed by a sum of squares of the amplitudes of the fast axis component f′ and the slow axis component s′ reflected by the anisotropic object 53 as shown in the lower diagram of FIG. 21. In other words, second intensity Iₒₚₑₙ² detected in a case of carrying out one nicol observation of the anisotropic object 53 is as follows.

Iₒₚₑₙ²=(Rf×f′)²+(Rs×s′)²=Rf²I² cos²(φ)+Rs²I² sin²((φ)=Rs²×I²((Rf²/Rs²)cos²(φ)+sin²(φ))≈Rs²I² sin²(φ)

In the last approximation, a case where Rf is sufficiently smaller than Rs (Rf<<Rs) is assumed. Therefore, the second intensity Iₒₚₑₙ² changes in proportion to sin²(φ) (Iₒₚₑₙ²∝sin²(φ)) and a periodic function that fluctuates with a cycle of π with respect to the angle φ. It should be noted that the angle φ can be replaced by the incident polarization angle θ and the phase component θ₀. Therefore, the second intensity Iₒₚₑₙ² fluctuates with a cycle of π also with respect to the incident polarization angle θ. If Rf is closer to Rs, it indicates that it is difficult to detect the anisotropy with the one nicol because φ dependency of the intensity is small.

As described above, the second intensity detected in a case of carrying out one nicol observation of the anisotropic object 53 fluctuates with a fluctuation cycle different from that of the first intensity detected in crossed nicols observation. The process of determining a quadrant including the fiber direction 56 of the anisotropic object 53 is performed utilizing this fluctuation cycle difference.

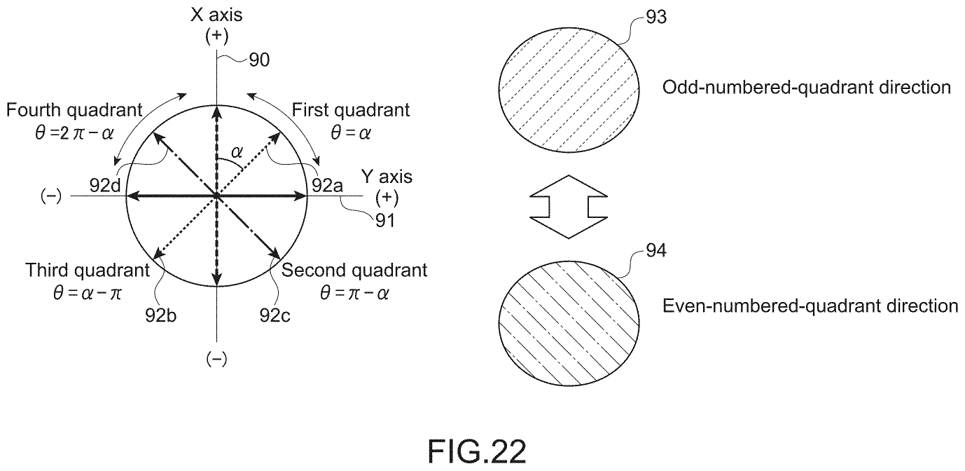

FIG. 22 is a schematic view for describing a quadrant including the fiber direction 56 of the anisotropic object 53. The X-axis 90 and the Y-axis 91 orthogonal to each other is shown on the left-hand side of FIG. 21. The X-axis 90 and the Y-axis 91 are set to be parallel to the upper and lower directions 71 and the left and right directions 72 of the observation image 73 (the imaging range 70 of the image sensor 32). It should be noted that in this embodiment, the upper and lower directions 71 of the imaging range 70 correspond to a reference direction that is a reference of the orientation direction. In addition, the left and right directions 72 of the imaging range 70 correspond to an orthogonal direction orthogonal to the reference direction.

Hereinafter, as shown in FIG. 22, it is assumed that a region between a positive direction (the upper direction) of the X-axis 90 and a positive direction (the right direction) of the Y-axis 91 is the first quadrant. In addition, it is assumed that a region between a negative direction (the lower direction) of the X-axis 90 and the positive direction (the right direction) of the Y-axis 91 is the second quadrant. In addition, it is assumed that a region between the negative direction (the lower direction) of the X-axis 90 and a negative direction (the left direction) of the Y-axis 91 is the third quadrant. In addition, it is assumed that a region between the positive direction (the upper direction) of the X-axis 90 and the negative direction (the left direction) of the Y-axis 91 is the fourth quadrant.

For example, as shown in FIG. 22, it is assumed that the phase component θ₀ is calculated as α(0≤α≤π/2) in crossed nicols observation. In this case, the direction indicated by θ=α or the direction orthogonal to that direction is the fiber direction 56 of the anisotropic object 53.

As shown in FIG. 22, the direction 92a indicated by θ=α is included in the first quadrant. This direction 92a included in the first quadrant and the direction 92b indicated by expressed by θ=α−π included in the third quadrant express the same fiber direction 56. In addition, the direction 92c indicated by θ=π−α orthogonal to the direction 92a indicated by θ=α is included in the second quadrant. Also in this case, the direction 92c included in the second quadrant and the direction 92d indicated by θ=2π−α included in the fourth quadrant represent the same fiber direction 56.

That is, as shown on the right-hand side of FIG. 22, the fiber direction 56 is included in either one of an even-numbered quadrant 93 (the first quadrant and the third quadrant) or an odd-numbered quadrant 94 (the second quadrant and the fourth quadrant). Therefore, in the process of determining a quadrant including the fiber direction 56 of the anisotropic object 53, it is unnecessary to determine which quadrant of the first to fourth quadrants it is included in. It is only necessary to determine which one of the even-numbered quadrant 93 and the odd-numbered quadrant 94 it is included in. In this embodiment, the even-numbered quadrant 93 and the odd-numbered quadrant 94 are included in a quadrant defined by a reference direction that is a reference of the orientation direction and an orthogonal direction orthogonal to the reference direction.

In this embodiment, the analysis section 43 determines a quadrant including the fiber direction 56 (the orientation direction) of the anisotropic object 53. In other words, the analysis section 43 performs a determination process of determining which one of the even-numbered quadrant 93 or the odd-numbered quadrant 94 the fiber direction 56 of the anisotropic object 53 is included in.

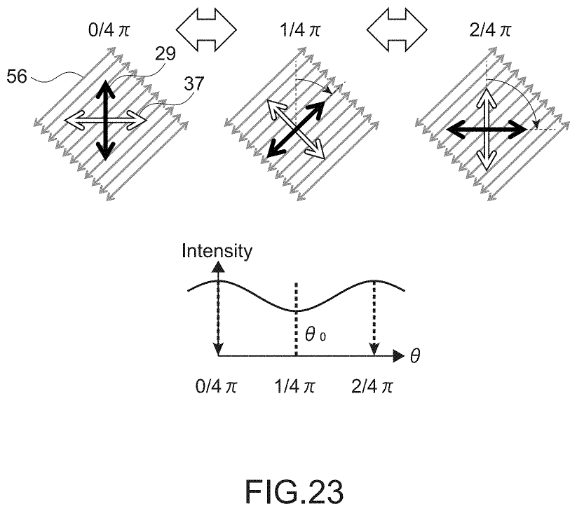

FIG. 23 is a diagram showing an example of the first intensity detected in a case of carrying out crossed nicols observation of the anisotropic object 53. For example, it is assumed that crossed nicols observation of the anisotropic object 53 is carried out and a change in first intensity as shown in the graph of FIG. 23 is observed. The phase component θ₀ calculated on the basis of the change in first intensity indicates, as described above, the direction parallel or orthogonal to the fiber direction 56 of the anisotropic object 53. In the determination process, the quadrant including the direction (the fiber direction 56) indicated by this the phase component θ₀ is determined in one nicol observation.

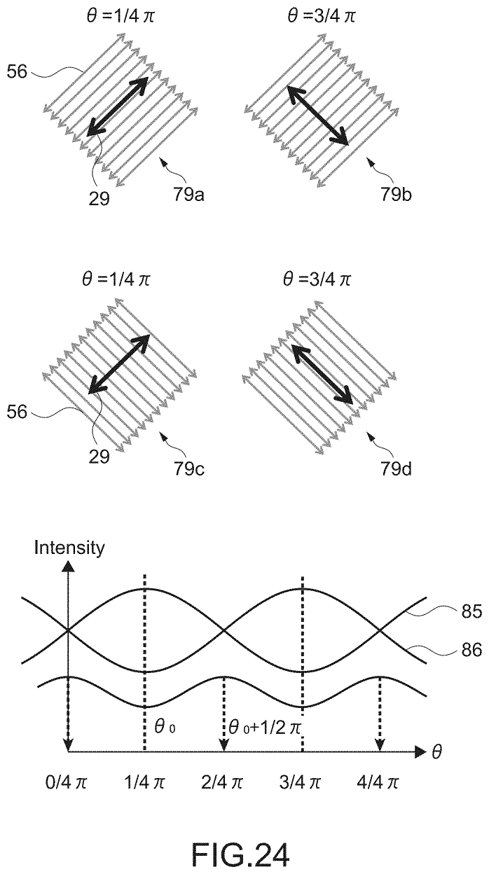

FIG. 24 is a diagram for describing an example of the determination process of the quadrant including the fiber direction 56. The upper diagram of FIG. 24 is a schematic view showing the relation between the first polarization direction 29 and the fiber direction 56 in one nicol observation. FIG. 24 schematically shows the state 79a (79c) of θ=π/4 and the state 79b (79d) of θ=¾π in a case where the angle between the fiber direction 56 and the upper and lower directions 71 is π/4 (¾π).

The graph of FIG. 24 shows first data 85 and second data 86 showing a change in the second intensity detected in the case of observing the anisotropic object 53 by one nicol observation. The first data 85 indicates a change in the second intensity in a case where the angle between the fiber direction 56 and the upper and lower directions 71 is π/4 (45°). The second data 86 indicates a change in the second intensity in a case where the angle between the fiber direction 56 and the upper and lower directions 71 is ¾π (135°).

As described above with reference to FIG. 21, the second intensity is a periodic function that fluctuates with a cycle of π (180°) with respect to the incident polarization angle θ. Therefore, as shown in the graph of FIG. 24, the first data 85 and the second data 86 fluctuate with the cycle π. In addition, the fiber direction 56 of the anisotropic object 53 in which the first data 85 and the second data 86 are detected are orthogonal to each other. Therefore, a deviation of the phase of vibration indicated by the respective data is 90°.

In a case where the angle between the fiber direction 56 and the upper and lower directions 71 is π/4, i.e., in a case where the fiber direction 56 is included in the even-numbered quadrant 93, the first data 85 takes a peak value in the state 79a of θ=π/4 and takes a bottom value in the state 79b of θ=¾π. In addition, in a case where the angle between the fiber direction 56 and the upper and lower directions 71 is ¾π, i.e., in a case where the fiber direction 56 is included in the odd-numbered quadrant 94, the first data 85 takes a bottom value in the state 79c of θ=π/4 and takes a peak value in the state 79d of θ=¾π. As described above, in one nicol observation, a change in the second intensity according to rotation of the first polarization direction differs in a case where the quadrant including the fiber direction 56 is the even-numbered quadrant 93 and in a case where the quadrant including the fiber direction 56 is the odd-numbered quadrant 94.

For example, it is assumed that the rotation control section 41 has rotated the first polarization direction 29 by a predetermined angle ΩZ. In this case, in accordance with rotation by the predetermined angle Ω, the second intensity changes along the first data 85 or the second data 86. The analysis section 43 determines whether the second intensity has changed along either one of the first data 85 or the second data 86. Accordingly, the quadrant including the fiber direction 56 can be determined. The determination result is stored as the information regarding the fiber direction which is the biological tissue information.

It should be noted that the second intensity changes in accordance with the value of the predetermined angle Ω. Therefore, the amount of increase/decrease of the second intensity and the like can be controlled by setting the predetermined angle Ω as appropriate. The details of the predetermined angle Ω will be described later.

As described above, in this embodiment, the rotation control section 41 rotates the first polarization direction by the predetermined angle Ω. Then, the analysis section 43 calculates the information regarding the fiber direction 56 of the anisotropic object 53 included in the observation target 1 on the basis of a change in the second intensity according to rotation of the first polarization direction 29 by the predetermined angle Ω.

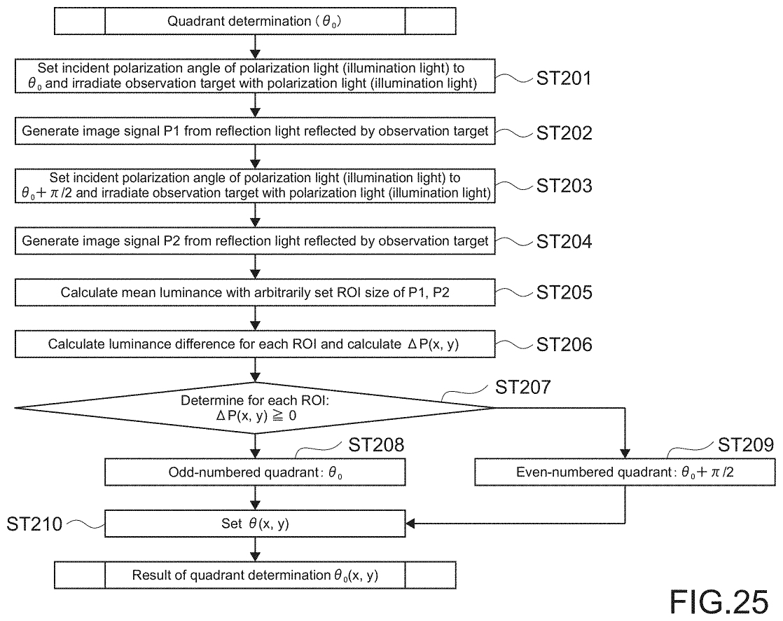

FIG. 25 is a flowchart showing an example of the determination process of the quadrant including the fiber direction 56. When execution of the process of determining a quadrant is started (Step 109 of FIG. 12) as shown in FIG. 25, the imaging system 30 is shifted to the configuration for performing one nicol observation. In other words, the second polarization element 31 is removed from the optical path of the reflection light 4 from the observation target 1 (see FIG. 20).

First of all, the rotation control section 41 sets the incident polarization angle θ of the first polarization direction 29 to a start state of the phase component θ₀ and the polarization light 3 of the first polarization direction is emitted to the observation target 1 (Step 201). Then, the image sensor 32 generates an image signal P1 according to the reflection light 4 from the observation target (Step 202).

As shown in the graph of FIG. 24, the state (start state) of θ=θ₀ is a state in which the second intensity is a peak value (the first data 85) or a bottom value (the second data 86). In this embodiment, the start state corresponds to a predetermined state set on the basis of a change in the first intensity.

It should be noted that the first data 85 is a bottom value and the second data 86 is a peak value in a state in which the first polarization direction 29 is rotated by ±π/2 from the state of θ=θ₀. Therefore, the amount of change in the second intensity is maximum irrespective of the quadrant including the fiber direction 56 in a state in which the first polarization direction 29 is rotated by ±π/2 from the start state.

The rotation control section 41 rotates the first polarization direction by the predetermined angle Ω from the start state. In this embodiment, the predetermined angle is set to ±90° (±π/2). As a result, a change in the second intensity is maximum and a change in the second intensity can be detected with high accuracy. It should be noted that in FIG. 25, +π/2 is used as the predetermined angle Ω. Therefore, the first polarization direction 29 is set such that the incident polarization angle θ=θ₀+π/2 is established.

The polarization light 3 of the first polarization direction is emitted to the observation target 1 at the incident polarization angle θ=θ₀+π/2 (Step 203). The image sensor 32 generates an image signal P2 according to the reflection light 4 from the observation target (Step 204).

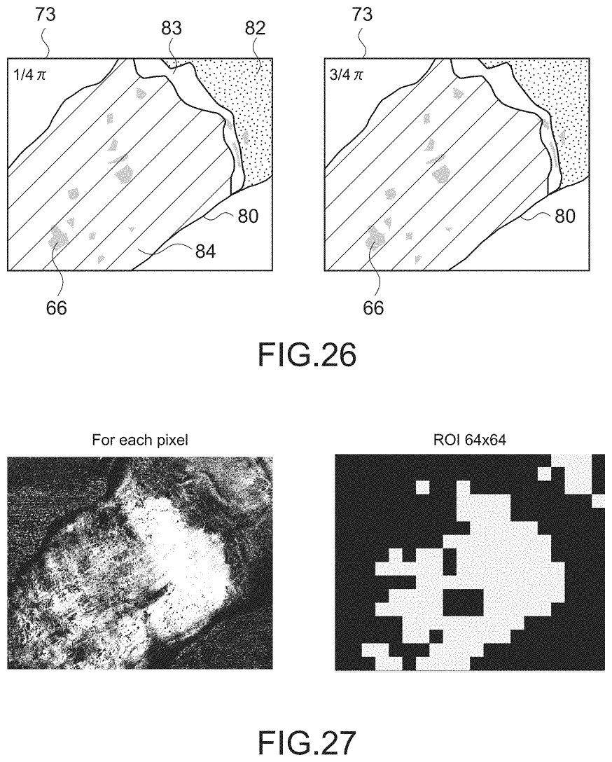

FIG. 26 is a schematic view showing an example of the image of the observation target 1 imaged in one nicol observation. A schematic view of the observation image 73 constituted by the image signal P1 is shown on the left-hand side of FIG. 26. In addition, a schematic view the observation image 73 constituted by the image signal P2 is shown on the right-hand side of FIG. 26.

It should be noted that in a case of imaging the observation target 1 in one nicol observation, specular reflection components reflected by the surface of the observation target 1 are sometimes detected. In the schematic views on the right- and left-hand sides of FIG. 26, regions in which strong specular reflection is caused are schematically shown as gray regions 66.

The analysis section 43 calculates the mean luminance in the ROI for each of ROIs, into which the observation image 73 constituted by the image signal P1 is divided (Step 205). This mean luminance corresponds to an average value of the second intensity detected in the ROI. Information regarding the calculated mean luminance for each ROI is saved as an image signal P1′. Similarly, also regarding the observation image 73 constituted by the image signal P2, the mean luminance for each ROI is calculated and an image signal P2′ is saved.

A difference of the mean luminance is calculated and a difference image signal ΔP(x, y) is calculated for each ROI of the image signals P1′ and P2′ (Step 206). Specifically, the mean luminance (the image signal P2′) in a case where θ=θ₀+π/2 is established is subtracted from the mean luminance (the image signal P1′) in a case where θ=θ₀ is established. Therefore, a change in the mean luminance (the average value of the second intensity) of each ROI detected in a case where the incident polarization angle θ is θ₀ and θ₀+π/2 is stored as the difference image signal ΔP(x, y). It should be noted that x and y are parameters indicating the position of each ROI.

On the basis of the difference image signal ΔP(x, y), the quadrant determination is performed for each ROI (Step 207). The following condition equation is used for quadrant determination.

ΔP(x, y)≥0

With respect to a certain ROI, it is determined that ΔP(x, y) is 0 or more (Yes of Step 207). In this case, as shown in the graph of FIG. 24, it can be considered that a peak value was detected in a case where θ=θ₀ is established and a bottom value was detected in a case where θ=θ₀+π/2 is established. Therefore, with respect to the ROI whose ΔP(x, y) is determined to be 0 or more, a quadrant including the fiber direction 56 of the anisotropic object 53 included in that ROI is set as the even-numbered quadrant 93 (Step 208).

On the other hand, in a case where it is determined that ΔP(x, y) is smaller than 0 (minus) (Yes of Step 207), the quadrant including the fiber direction 56 of the anisotropic object 53 included in the ROI is set as the odd-numbered quadrant 94 (Step 208).

FIG. 27 is a diagram showing a process result of the determination process of the quadrant including the fiber direction 56. A process result in a case where a quadrant determination is performed for each pixel is shown on the left-hand side of FIG. 27. A result similar to that in a case where the size of the ROI is set to 1 pixel×1 pixel is obtained. In addition, a process result in a case where the size of the ROI is set to 64 pixels×64 pixels is shown on the right-hand side of FIG. 27.

In each process result of FIG. 27, the ROI (the pixel) in which the fiber direction 56 of the anisotropic object 53 is determined to be included in the even-numbered quadrant 93 is displayed in bright color. As shown in FIG. 27, in the ROI corresponding to the muscle layer 84 of the observation target 1, the fiber direction 56 of the muscle layer 84 (the anisotropic object 53) is determined to be included in the even-numbered quadrant 93. In other words, the fiber direction 56 is determined to be a direction indicated by the phase component 00 (substantially π/4).

On the basis of the determination result for each ROI, an optical axis direction representing the fiber direction 56 of the anisotropic object 53 included in the ROI is set (Step 210). The optical axis direction of the anisotropic object 53 is an angle indicating the directions of the slow axis 55 and the fast axis 54 of the anisotropic object 53. In this embodiment, an angle indicating the direction of the slow axis 55, i.e., the fiber direction 56 is set as the optical axis direction.

For example, it is assumed that it is determined that the fiber direction 56 is included in the even-numbered quadrant 9. In this case, the phase component θ₀ is an angle in a range of 0≤θ₀<90, and thus the direction indicated by the phase component θ₀ is the fiber direction 56 as it is. In other words, the angle between the fiber direction 56 and the upper and lower directions 71 of the observation image 73 is indicated by the phase component θ₀. In addition, for example, in a case where it is determined that the fiber direction 56 is included in the odd-numbered quadrant 94, for example, the fiber direction 56 is the direction orthogonal to the direction indicated by the phase component θ₀. In this case, the angle between the fiber direction 56 and the upper and lower directions 71 of the observation image 73 is indicated by the phase component θ₀+π/2.

As described above, the analysis section 43 calculates an angle between the fiber direction 56 and the upper and lower directions 71 of the observation image 73. The calculated angle is set as the optical axis direction. The process of setting the optical axis direction is performed for each ROI. Hereinafter, the optical axis direction will be referred to as the optical axis direction θ₀ with the same reference sign as the phase component θ₀. In this embodiment, the optical axis direction 00 corresponds to the orientation angle.

The optical axis direction θ₀ set for each ROI is used for the processes after Step 108 shown in FIG. 12 as a quadrant determination result θ₀ (x, y). In other words, on the basis of the quadrant determination result, the analysis section 43 generates an image on which the fiber direction 56 and the like included in each ROI has been mapped. The image is displayed on the display unit 50.

As described above, the endoscopic device 100 according to this embodiment irradiates the observation target 1 with the polarization light 3 of the first polarization direction 29. Among beams of reflection light that are reflected by the observation target 1, the polarization component 5 of the second polarization direction 37 that intersects with the first polarization direction 29 is extracted. The first polarization direction 29 and the second polarization direction 37 are rotated while the intersection angle is maintained, and biological tissue information is calculated on the basis of a change in intensity of the polarization component 5 according to the rotation operation. Accordingly, the observation target 1 can be observed in detail.

As a method of emitting the polarized light and observing the biological tissue, a method of identifying the fibrous structure and the nonfibrous structure included in the biological tissue is conceivable. In this method, it is possible to identify the position, the region, and the like in which the fibrous structure which is the anisotropic object 53 is included. On the other hand, only by identifying the fibrous structure and the nonfibrous structure, it may be difficult to observe the characteristics and the like of the anisotropic object 53.

In this embodiment, crossed nicols observation of the observation target 1 is performed by rotating the first polarization direction 29 and the second polarization direction 37. The analysis section 43 analyzes a change according to the rotation operation of the first intensity detected in crossed nicols observation and calculates the biological tissue information related to the observation target 1.

By analyzing a change in first intensity, the presence/absence of the anisotropic object 53 can be determined with high accuracy. Accordingly, the fibrous structure 57 and the non-fibrous structure 58 can be identified with high accuracy. As a result, when the tumor and the like are resected using endoscopic submucosal dissection (ESD), exposure of the circular muscle layer due to unintended perforation and the like can be identified with high accuracy. As a matter of course, not limited to the ESD, the present technology may be used for a procedure such as endoscopic mucosal resection (EMR).

In addition, in this embodiment, the quadrant including the fiber direction 56 of the anisotropic object 53 is determined by also using one nicol observation. In other words, the relative fiber direction 56 calculated in crossed nicols observation can be handled as a direction also including a quadrant. Accordingly, the fiber direction 56, its boundary, and the like can be accurately observed. As a result, for example, the orientation and the like of muscle fibers that constitute muscle and the like can be observed in detail.

The biological tissue information calculated by the analysis section 43 includes information regarding the orientation and the anisotropy. Therefore, for example, the orientation of the anisotropic object 53 or the anisotropy and the like can be mapped. As a result, degradation of muscle fibers inside muscle, abnormal orientation of cardiac muscle cells in hypertrophic cardiomyopathy, or a necrosis part of cardiac muscle due to coronary stenosis can be visualized. As described above, degradation, a lesion, or the like in the anisotropic object 53 constituted by the structure (the fibrous structure 57) can be observed in detail.

### Second Embodiment

An observation device according to a second embodiment of the present technology will be described. Hereinafter, descriptions of portions similar to the configurations and actions in the endoscopic device 100 described in the above-mentioned embodiment will be omitted or simplified.

In the above-mentioned embodiment, one nicol observation is performed on the observation target 1 and the quadrant including the fiber direction 56 of the anisotropic object 53 is determined. In this embodiment, the process of calculating the fiber direction 56 of the anisotropic object 53 is performed on the basis of the observation result of one nicol observation.

In a case where the anisotropic object 53 is observed in one nicol observation, the intensity (the second intensity) of the reflection light 4 which is detected fluctuates with a cycle of π (see the graph of FIG. 24). The fiber direction 56 of the anisotropic object 53 is calculated by analyzing a change in intensity of the reflection light 4 expressed by this fluctuation with the cycle of π.

For example, in the first data 85 shown in FIG. 24, the incident polarization angle θ (π/4) at which the first data 85 takes a peak value corresponds to the angle (the optical axis direction θ₀) indicating the fiber direction 56 of the anisotropic object 53. In addition, regarding the second data 86, the incident polarization angle θ (¾π) at which the second data 86 takes a peak value corresponds to the optical axis direction θ₀.

Therefore, the optical axis direction θ₀ of the anisotropic object 53, i.e., the fiber direction 56 of the anisotropic object 53 can be calculated by calculating the incident polarization angle θ at which the second intensity takes a peak value. As described above, the fiber direction 56 of the anisotropic object 53 can be directly calculated in one nicol observation.

As the process of calculating the fiber direction 56 of the anisotropic object 53, the fitting process and the like using the periodic function (sin²(θ) and the like) indicating a change in the second intensity, for example, is performed. Accordingly, the optical axis direction θ₀ of the anisotropic object 53 (the incident polarization angle θ at which a peak value is obtained) can be calculated with high accuracy. Besides, the process of calculating the fiber direction 56 is not limited and any method may be used.

It should be noted that the configuration capable of performing one nicol observation, i.e., the configuration capable of detecting the intensity of the reflection light 4 that fluctuates with a cycle of π is not limited to the configuration shown in FIG. 20 and other configurations may be used.

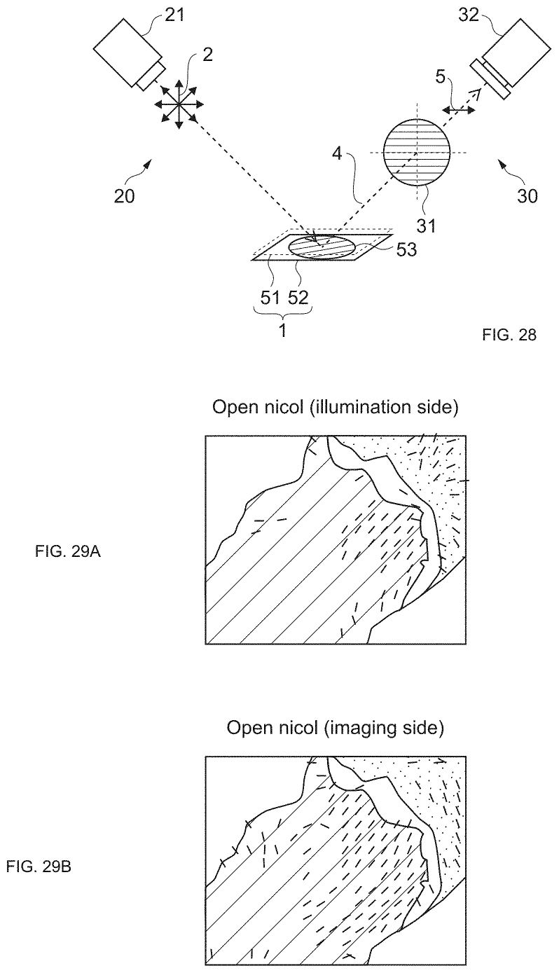

FIG. 28 is a schematic view showing another configuration example for performing one nicol observation. The configuration shown in FIG. 28 is a configuration obtained by removing the first polarization element 22 (the polarizing plate 28 having the first polarization axis 27) from the configuration for crossed nicols observation described in FIGS. 2 and 4 or the like.

As shown in FIG. 28, the illumination light 2 which is non-polarized light without the particular polarization direction is emitted to the observation target 1 from the light source 21. In this embodiment, a fourth polarization section that emits the non-polarized light to the biological tissue is realized by removing the first polarization element 22 from the illumination system 20. It should be noted that the second polarization element 31 is used in a manner similar to that in crossed nicols observation.

The illumination light 2 emitted from the illumination system 20 is reflected by the observation target 1. This reflection light 4 is incident on the second polarization element 31. The second polarization element 31 extracts the polarization component 5 of the second polarization direction among beams of the reflected illumination light 2. The polarization component 5 of the second polarization direction is incident on the image sensor 32. The image sensor 32 generates an image signal on the basis of the polarization component 5 that has been incident thereon and outputs that image signal to the intensity detection section 42.

As described above, the image sensor 32 and the intensity detection section 42 detects the third intensity which is the intensity of the polarization component 5 of the second polarization direction extracted by the second polarization element 31 among beams of the non-polarized light reflected by the observation target 1. In other words, it can also be said that the third intensity is the intensity of the reflection light 4 detected in one nicol observation using the second polarization element 31. It should be noted that one nicol observation using the second polarization element 31 corresponds to observation performed in a state in which the fourth polarization section has been configured. It should be noted that a method of realizing the fourth polarization section is not limited and any method may be used.

In a case of rotating the second polarization direction 37 and observing the anisotropic object 53, the detected intensity (the third intensity) of the reflection light 4 changes in a manner similar to that of the second intensity (the first data 85 or the second data 86) indicated by the graph of FIG. 24, for example. In other words, the third intensity fluctuates with a cycle of π with respect to rotation of the second polarization direction 37.

For example, it is assumed that the second polarization direction 37 is rotated by the predetermined angle Ω′. In this case, the quadrant including the fiber direction of the anisotropic object 53 can be determined on the basis of a change in the third intensity detected in accordance with rotation by the predetermined angle Ω′. In addition, for example, in a case where data indicating a change in the third intensity is generated, the angle (the optical axis direction θ₀) and the like indicating the fiber direction 56 of the anisotropic object 53 can be calculated by performing the fitting process and the like with respect to the generated data.

As described above, in one nicol observation performed using the configuration shown in FIG. 28, the rotation control section 41 rotates the second polarization direction by the predetermined angle Ω′. Then, the analysis section 43 calculates information regarding the fiber direction 56 of the anisotropic object 53 included in the observation target 1 on the basis of a change in the third intensity according to rotation of the second polarization direction by the predetermined angle Ω′. It should be noted that the method or the like of setting the predetermined angle Ω′ is not limited and may be set as appropriate such that the information regarding the fiber direction 56 can be calculated with desired accuracy, for example.

It should be noted that FIG. 28 schematically shows the reflection light 4 reflected by the anisotropic object 53 in the inside 52 of the observation target 1. In actual observation, the reflection light 4 reflected by the observation target 1 includes specular reflection components on the observation target 1, components reflected by the non-fibrous structure 58, and the like. Therefore, the third intensity includes the intensity of the reflection light 4 and specular reflection intensity due to the anisotropic object 53 and the non-fibrous structure 58.

As described above, also in a case of using the configuration from which the first polarization element 22 has been removed, the third intensity that fluctuates with a cycle of π can be detected. In other words, one nicol observation can be performed irrespective of which of the configuration (the configuration of FIG. 20) obtained by removing polarization element (the second polarization element 31) of the imaging system 30 has been removed from the configuration in which crossed nicols observation and the configuration (the configuration of FIG. 28) obtained by removing the polarization element (the first polarization element 22) of the illumination system 20 is used.

Hereinafter, one nicol observation performed with the configuration obtained by removing the second polarization element 31, i.e., a configuration using the first polarization element 22 of the illumination system 20 will be referred to as one nicol observation on an illumination side. In addition, one nicol observation performed with the configuration obtained by removing the second polarization element 31, i.e., a configuration using the second polarization element 31 of the imaging system 30 will be referred to as one nicol observation on the imaging side.

FIG. 29 shows a result of detection of the fiber direction 56 using one nicol observation. FIG. 29A shows the fiber direction 56 calculated by one nicol observation on the illumination side (the configuration of FIG. 20). FIG. 29B shows the fiber direction 56 calculated by one nicol observation on the imaging side (the configuration of FIG. 28).

As shown in FIGS. 29A and 29B, at each position (each ROI 74) of the muscle layer 84 which is the anisotropic object 53, the fiber direction 56 substantially inclined by π/4 with respect to the upper and lower directions 71 is calculated. Therefore, it can be said that the muscle fiber direction is suitably observed irrespective of which of one nicol observation the illumination side or one nicol observation on the imaging side is used.

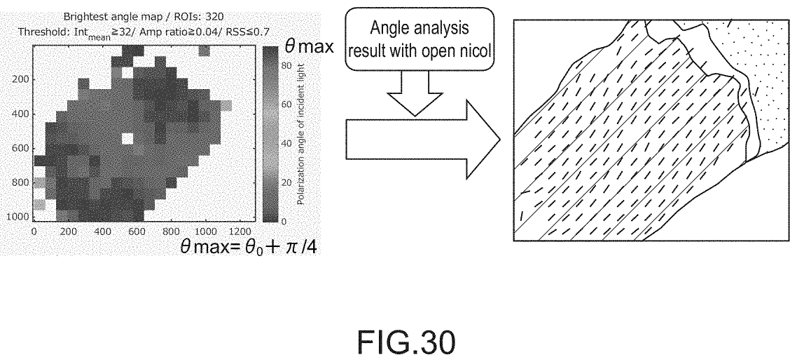

FIG. 30 is a diagram showing an example of the calculation process of the fiber direction 56 using detection results of crossed nicols observation and one nicol observation. The left-hand diagram of FIG. 30 is a diagram showing an example of information regarding the fiber direction 56 calculated using crossed nicols observation. The left-hand diagram of FIG. 30 shows a result of mapping the incident polarization angle θ=θₘₐₓ at which the luminance value in each ROI takes a peak value in a manner similar to that of FIG. 17. In addition, the right-hand diagram of FIG. 30 shows the fiber direction 56 calculated using crossed nicols observation and one nicol observation.

In this embodiment, as shown in FIG. 30, the process of calculating the fiber direction 56 of the anisotropic object 53 is performed by using the information regarding the fiber direction 56 calculated using crossed nicols observation and the fiber direction 56 (an angle analysis result) calculated using one nicol observation. The process of calculating the fiber direction 56 using crossed nicols observation and one nicol observation is not limited. Any process using information calculated in each type of observation may be performed.

It should be noted that as the angle analysis result, the result (FIG. 29A) in one nicol observation on the illumination side may be used or the result (FIG. 29B) in one nicol observation on the imaging side may be used.

In crossed nicols observation, the anisotropic object 53 can be accurately observed with small influence of specular reflection and the like. On the other hand, in one nicol observation, the optical axis direction θ₀ of the anisotropic object 53 can be directly calculated. Therefore, the fiber direction of the anisotropic object 53 can be calculated with sufficiently high accuracy by using the calculated optical axis direction θ₀ in one nicol observation in addition to the information regarding the fiber direction calculated in crossed nicols observation. As a result, the fiber direction of the biological tissue and the like can be observed in detail.

### Third Embodiment

In this embodiment, the threshold process regarding the intensity of the reflection light 4 detected in one nicol observation is performed and the fiber direction 56 of the anisotropic object 53 is calculated on the basis of a result of threshold process. This threshold process can be applied to both of one nicol observation on the illumination side and one nicol observation on the imaging side.

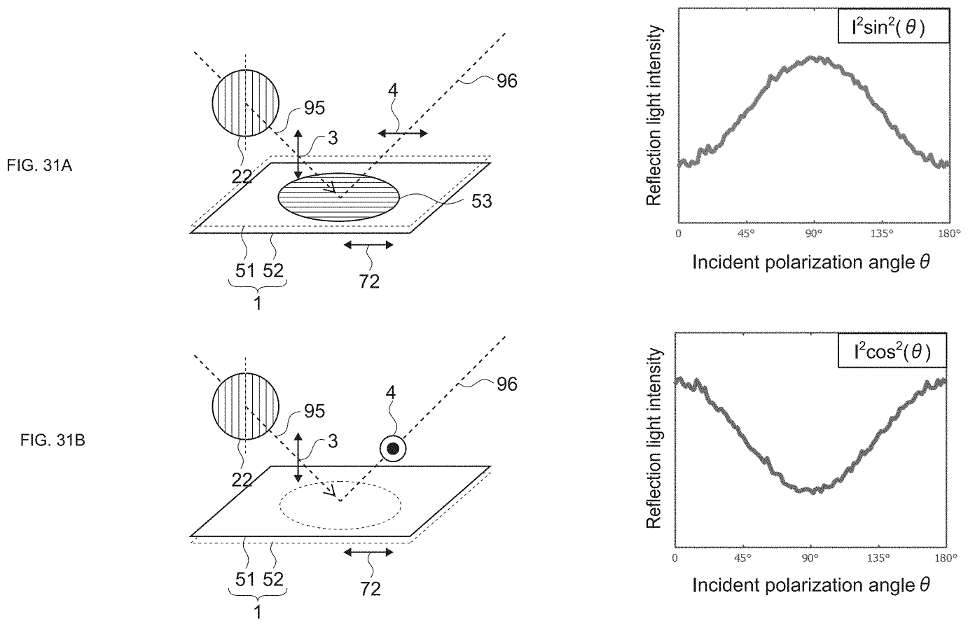

FIG. 31 is a diagram for describing reflection in one nicol observation on the illumination side. The right-hand diagram of FIG. 31A is a schematic view showing an example of reflection by the anisotropic object 53 in one nicol observation on the illumination side. The graph of FIG. 31A is a graph of the second intensity in a case where components of the reflection light 4 from the anisotropic object 53 is larger than components of other reflection light.

In one nicol observation, the polarization light 3 in the same orientation as the fiber direction 56 of the anisotropic object 53 is reflected most strongly. In the example shown in FIG. 31A, the direction of the fast axis 54 of the anisotropic object 53 is set to the upper and lower directions 71 of the imaging range 70 and the direction of the slow axis 55 (the fiber direction 56) is set to the left and right directions 72. In such arrangement, the second intensity changes in proportion to sin²(θ). Therefore, as shown in the graph of FIG. 31A, the second intensity detected in a case of carrying out one nicol observation of the anisotropic object 53 is maximum at the incident polarization angle θ=90°.

The right-hand diagram of FIG. 31B is a schematic view showing an example of specular reflection in one nicol observation on the illumination side. The graph of FIG. 31B is a graph of the second intensity in a case where specular reflection components on the surface of the observation target 1 are larger than components of other reflection light. In general, in specular reflection, the S-polarized light components perpendicular to an incident plane are reflected strongly. Here, the incident plane is a plane including an optical path 95 of the polarization light 3 and an optical path 96 of the reflection light 4, which are incident on the anisotropic object 53, and is a direction parallel to the upper and lower directions 71 of the imaging range 70 in the example shown in FIG. 31. It should be noted that in the right-hand diagram of FIG. 31B, the direction perpendicular to the incident plane is schematically indicated as the circle mark.

In a case where specular reflection components are dominant as shown in the graph of FIG. 31B, the second intensity is maximum in a state (θ=0° or 180°) in which the first polarization direction 29 is perpendicular to the incident plane. In addition, the second intensity is minimum in the state (θ=90°) in which the first polarization direction 29 is parallel to the incident plane. At this time, the second intensity is in proportion to cos²(θ).

As described above, in a case where specular reflection components are large in one nicol observation on the illumination side, the second intensity can change with a cycle of π (180°) with respect to the incident polarization angle θ. Therefore, it may be difficult to suitably calculate the fiber direction 56 of the anisotropic object 53 in a state in which specular reflection components are large.

It should be noted that the contents described in FIG. 31 also apply to a case where the third intensity is detected by performing one nicol observation on the imaging side. Hereinafter, the second and third intensity calculated in one nicol observation on the illumination side and the imaging side will be referred to as the detection intensity of one nicol observation.

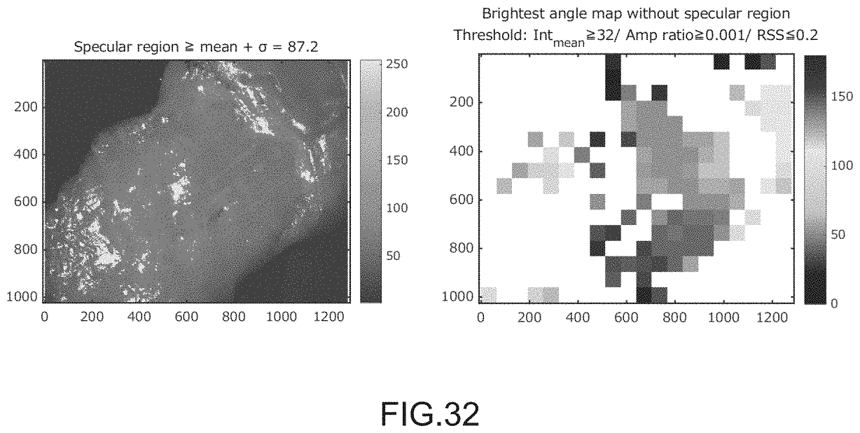

FIG. 32 is a diagram showing an example of the threshold process with respect to the detection intensity of one nicol observation. The left-hand diagram of FIG. 32 is a diagram showing a result of mapping of the detection intensity of one nicol observation. The region displayed in bright color is a region in which the detection intensity is high.

In general, the luminance of the reflection light 4 (specular reflection components) reflected by the surface of the observation target 1 is larger than the luminance of the reflection light 4 reflected by the inside and the like of the observation target 1. Therefore, the region displayed in a bright state in the right-hand diagram of FIG. 32 can be considered as a region in which it is highly likely that specular reflection has been detected.

In this embodiment, the first threshold related to the luminance (detection intensity) detected in one nicol observation is set. Then, it is determined whether or not the detection intensity of one nicol observation is equal to or lower than the first threshold. Accordingly, a region in which specular reflection components are large and other regions can be identified.

The first threshold is set to the value (Iₘₑₐₙ+σ) obtained by adding dispersion σ of a luminance distribution to the average value Iₘₑₐₙ of the luminance distribution (the average value of the luminance value of each pixel) on the observation target 1, for example. In other words, in a case where regarding the luminance value I, I≥Iₘₑₐₙ+σ is established, it is determined to be a region in which specular reflection components are large. This determination is performed for each pixel.

A threshold is set by using the luminance distribution on the observation target 1 as a reference as described above. In this manner, also in a case where the imaging condition and the like are changed, for example, it is possible to accurately detect a region in which specular reflection is strong. It should be noted that the method or the like of setting the first threshold is not limited and the first threshold may be set as appropriate such that a region in which specular reflection components are large can be suitably identified, for example.

In the right-hand diagram of FIG. 32, it is a map showing the fiber direction 56 in a case where the region in which specular reflection components are large has been excluded. When a pixel at which specular reflection is strong is determined, the proportion at which the pixel at which specular reflection is strong is included is calculated for each ROI 74 on the basis of the determination result. For example, the ROI 74 in which the proportion at which the pixel at which specular reflection is strong is included is higher than a predetermined proportion is excluded as the ROI 74 set to be the region in which specular reflection is strong. The predetermined proportion is set as appropriate such that the ROI in which specular reflection components are dominant can be suitably excluded, for example.

As shown in the right-hand diagram of FIG. 32, the ROI indicating the fiber direction 56 is not displayed with respect to the region (e.g., the lower left region of the figure) in which specular reflection components are large. Accordingly, a region in which specular reflection is sufficiently strong is excluded and the region or the like in which the reflection light 4 from the anisotropic object 53 is strong can be extracted and observed.

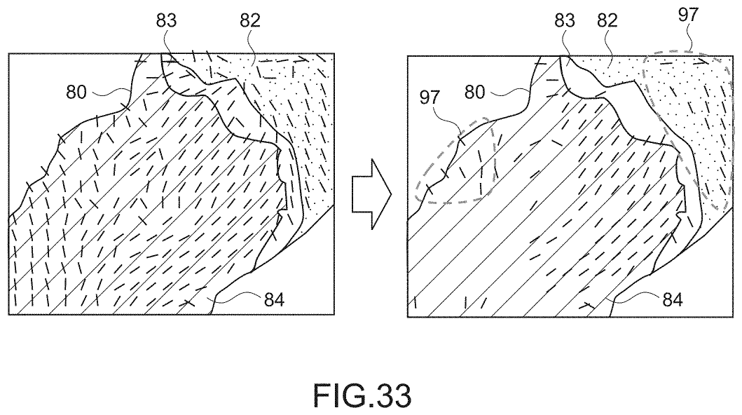

FIG. 33 is a diagram showing a result of the threshold process using the first threshold. The fiber direction 56 of each ROI 74 before the threshold process is performed is shown in the left-hand diagram of FIG. 33. In addition, the fiber direction 56 of each ROI 74 after the threshold process is performed is shown in the right-hand diagram of FIG. 33. It should be noted that the left- and the right-hand diagrams of FIG. 33 show results calculated on the basis of the detection intensity shown in the right-hand side of FIG. 32.

As shown in the figure on the left-hand side of FIG. 33, the direction substantially parallel to the upper and lower directions 71 is calculated in the region in which specular reflection is strong (e.g., the lower left region of the figure). In this manner, a direction different from the fiber direction 56 of the anisotropic object 53 is calculated with respect to the region in which specular reflection is strong, which can cause erroneous detection.

By performing the threshold process using the first threshold with respect to the detection intensity of one nicol observation, the ROI 74 in which the erroneous detection is caused is excluded. Accordingly, highly accurate observation can be realized by suitably detecting the fiber direction of the anisotropic object 53.

The timing at which the threshold process using the first threshold is performed is not limited. For example, as shown in FIG. 33, after the process of calculating the fiber direction 56 of the anisotropic object 53 with respect to each ROI 74 is performed, the threshold process using the first threshold may be performed. In addition, the fiber direction 56 of the anisotropic object 53 may be calculated after the ROI 74 in which specular reflection components are large is excluded in accordance with the threshold process, for example. Accordingly, the calculation amount can be reduced and the process time can be shortened.

It should be noted that in one nicol observation, for example, as in a region 97 surrounded with the dotted line in the right-hand diagram of FIG. 33, the detection intensity may be lower than the first threshold and the region in which specular reflection components are dominant may be observed. In such a region, for example, it can be considered that specular reflection on the nonfibrous structure or the like may be caused.

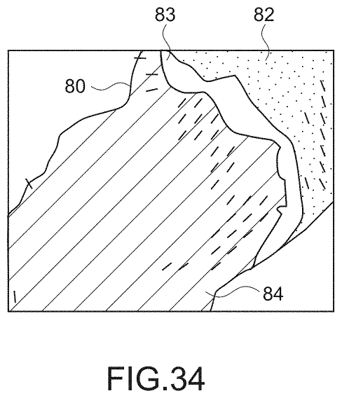

FIG. 34 is a diagram showing a result of another threshold process with respect to the detection intensity of one nicol observation. In this embodiment, the second threshold related to the amplitude of the detection intensity of one nicol observation is set and a threshold process using the second threshold is performed. The second threshold is set as appropriate such that specular reflection on the nonfibrous structure or the like and reflection on the anisotropic object 53 can be identified.

By performing the identification process using the second threshold as shown in FIG. 34, the ROI 74 set to the region 97 in which specular reflection on the nonfibrous structure or the like is caused is excluded. As a result, the ROI 74 in which the fiber direction 56 on the anisotropic object 53 has been suitably calculated can be extracted.

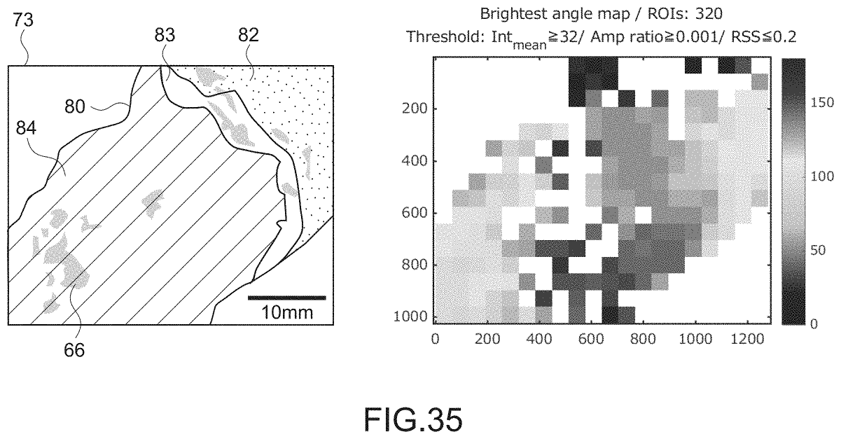

FIG. 35 is a diagram showing an example of a result of observation of the fiber direction 56 using one nicol observation shown as the comparative example. The left-hand diagram of FIG. 35 is a schematic view showing an example of the observation image 73 captured using one nicol observation and the region 66 in which specular reflection is strong is schematically shown as the gray region. In a case of calculating the fiber direction 56 of the anisotropic object 53 by using the detection intensity of one nicol observation as it is, there is a possibility that an erroneous angle is calculated as the fiber direction 56 in the region 66 in which specular reflection is strong as shown in the right-hand diagram of FIG. 35.

In this regard, in this embodiment, the erroneous detection of the fiber direction 56 is sufficiently suppressed by performing the threshold process related to the detection intensity of one nicol observation. In other words, as shown in the right-hand diagram of FIGS. 33 and 34 or the like, a suitable detection result can be displayed excluding the ROI 74 in which it is highly likely that the erroneous detection is caused. As a result, highly reliable observation using one nicol observation can be realized.

It should be noted that one nicol observation using the threshold process according to this embodiment may be performed alone. In other words, without performing crossed nicols observation, one nicol observation using the threshold process may be performed and the result of observation may be displayed as an emphasis image and the like. Accordingly, the observation time can be shortened and the usability of the apparatus is enhanced. As a matter of course, although one nicol observation using the threshold process is used, it may be performed together with crossed nicols observation as described in FIG. 30.

### Other Embodiments

The present technology is not limited to the above-mentioned embodiments and various other embodiments can be realized.

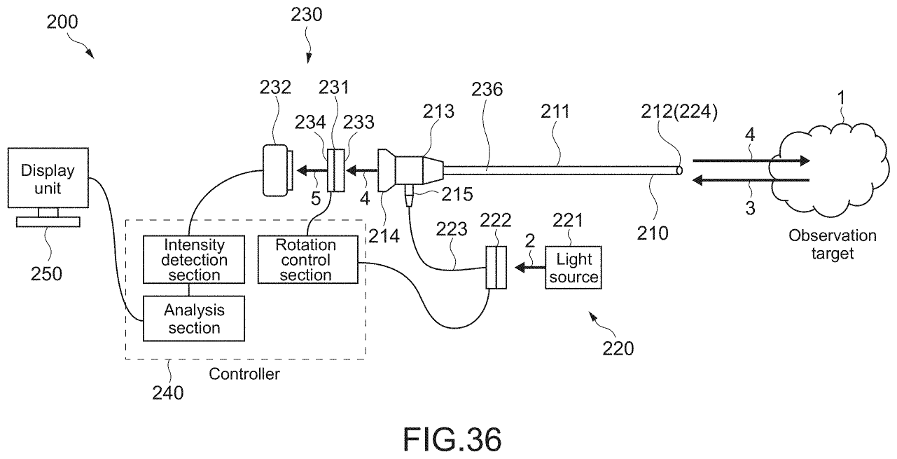

FIG. 36 is a diagram schematically showing a configuration example of an endoscopic device 200 that is an imaging device according to another embodiment of the present technology. The endoscopic device 200 includes an insertion unit 210, an illumination system 220, an imaging system 230, a controller 240, and a display unit 250. The endoscopic device 200 is configured as a rigid endoscope that is used for laparoscopic surgery or observation or the like of an otolaryngological area. It should be noted that the controller 240 and the display unit 250 shown in FIG. 36 are configured in a way similar to the controller 40 and the display unit 50 shown in FIG. 1.

The insertion unit 210 includes a rigid section 211, a tip section 212, and an operation section 213. The rigid section 211 has a thin tubular structure, and includes hard material such as stainless. The material, size, and the like of the rigid section 211 are not limited. They may be set as appropriate in accordance with its use purpose such as a surgery or observation.

The tip section 212 is provided at one end of the rigid section 211. The tip section 212 is inserted into an opening or the like made in an abdomen of a patient, and the tip section 212 reaches the vicinity of the observation target 1. Although not shown, the tip section 212 has an illumination opening, and an imaging opening. In addition, the tip section 212 may be appropriately provided with a nozzle or the like that is an outlet of water, air, or the like, a treatment tool outlet through which forceps or the like moves in and out, or the like.

The operation section 213 is provided at an end of the rigid section 211 opposite to the tip section 212. The operation section 213 includes a scope holder 214 and an optical port 215. A forceps port through which a treatment tool such as forceps moves in and out or the like may also function as the optical port 215, for example. In addition, the operation section 213 may be provided with a lever, a switch, or the like that is necessary to operate the insertion unit 210.

The illumination system 220 includes a light source 221, a first polarization element 222, a polarization maintaining fiber 223, and an illumination lens 224. The light source 221 and the first polarization element 222 are configured in ways similar to the light source 21 and the first polarization element 22 shown in FIG. 1. The polarization maintaining fiber 223 is inserted into the optical port 215 from the first polarization element 222, passes through the inside of the rigid section 211, and extends to the tip section 212. The illumination lens 224 is disposed in the illumination opening made in the tip section 212.

In the illumination system 220, the first polarization element 222 polarizes the illumination light 2 emitted from the light source 221 in the first polarization direction, and emits the polarized light to the observation target 1 via the polarization maintaining fiber 223 and the illumination lens 224.

The imaging system 230 includes a relay optical system 236, a second polarization element 231 and an image sensor 232. The relay optical system 236 is an optical system that connects the imaging opening to the scope holder 214, and is installed in the insertion unit 210. The relay optical system 236 is appropriately configured to be capable of maintaining a polarization direction of the reflection light 4. As shown in FIG. 8, the reflection light 4 reflected by the observation target 1 passes through the relay optical system 236 disposed in the insertion unit 210, and then is emitted.

The second polarization element 231 is disposed outside the scope holder 214. A liquid crystal polarizer including a liquid crystal variable wave plate 233 and a polarizing plate 234 is used as the second polarization element 231. As shown in FIG. 8, in the second polarization element 231, the liquid crystal variable wave plate 233 is disposed in such a manner that the liquid crystal variable wave plate 233 faces the scope holder 214.

The reflection light 4 that has emitted from the observation target 1 and passed through the relay optical system 236 is incident on the liquid crystal variable wave plate 233. The second polarization element 231 extracts a polarization component 5 of the second polarization direction from beams of the reflection light 4, and the polarizing plate 234 emits the extracted polarization component 5.

The image sensor 232 is provided on the opposite side of the scope holder 214 with the second polarization element 231 interposed therebetween. Therefore, the polarization component 5 of the second polarization direction extracted by the second polarization element 231 is incident on the image sensor 232.

As in the first embodiment, the endoscopic device 200 controls the first polarization element 222 and the second polarization element 231 and performs the crossed nicols observation (substantially crossed nicols observation). In addition, in a state in which either the first polarization element 222 or the second polarization element 231 has been removed, one nicol observation is performed and the process of determining a quadrant including the fiber direction of the anisotropic object is performed. Then, an emphasis image indicating the fiber direction of the anisotropic object included in the observation target 1, the orientation, the anisotropy, and the like is displayed on the display unit 250.

As described above, it is possible to perform the substantially crossed nicols observation even when using the endoscopic device 200 configured as the rigid endoscope. Therefore, it is possible to accurately detect the biological tissue. This makes it possible to observe the biological tissue in detail not only in a case where an area of gastroenterological medicine is observed by using a soft endoscope, but also in a case of laparoscopic surgery or observation or the like of an otolaryngological area.

In the above-described embodiments, the endoscopic devices 100 and 200 are configured as the observation devices. However, the observation device is not limited thereto. The observation device may be configured in a way different from the above-described embodiments. For example, a surgical microscope may be configured as the observation device. In other words, the surgical microscope including the first polarization element and the second polarization element may be appropriately configured. For example, it is possible to observe an optically anisotropic biological tissue (an anisotropic object) in detail by controlling rotation of the first and second polarization directions through the processes shown in FIGS. 12 and 25. This makes it possible to magnify and observe the anisotropic object, for example.

In addition, when a computer operated by the doctor or the like and another computer capable of communication via a network work in conjunction with each other, the observation method and the program according to the present technology are performed, and this makes it possible to configure the observation device according to the present technology.

That is, the observation method and the program according to the present technology can be performed not only in a computer system consisting of a single computer, but also in a computer system in which a plurality of computers cooperatively operates. It should be noted that in the present disclosure, the system means an aggregate of a plurality of components (devices, modules (parts), or the like) and it does not matter whether or not all the components are housed in a same casing. Therefore, a plurality of devices housed in separate casings and connected to one another via a network is treated as a system, and a single device including a plurality of modules housed in a single casing is also treated as a system.

The execution of the observation method and the program according to the present technology by the computer system include, for example, both of a case where control of rotation of the first and second polarization directions, calculation of biological tissue information, and the like are performed by a single computer and a case where those processes are performed by different computers. Further, the execution of the respective processes by predetermined computers includes causing another computer to perform some or all of those processes and acquiring results thereof.

That is, the observation method and the program according to the present technology are also applicable to a cloud computing configuration in which one function is shared and cooperatively processed by a plurality of devices via a network.

In addition, the present technology is applicable to observation devices and observation systems not only in medical/biological fields but also in various kinds of other fields.

At least two feature parts of the feature parts according to the present technology described above can be combined. That is, the various feature parts described in the embodiments may be arbitrarily combined irrespective of the embodiments. Further, various effects described above are merely examples and are not limited, and other effects may be exerted.

It should be noted that the present technology may also be configured as below.
(1) An observation device including:

a first polarization section that irradiates a biological tissue with polarization light of a first polarization direction;

a second polarization section that extracts a polarization component of a second polarization direction that intersects with the first polarization direction, from beams of reflection light that are the polarization light reflected by the biological tissue;

a rotation control section that rotates each of the first polarization direction and the second polarization direction such that an intersection angle between the first polarization direction and the second polarization direction is maintained; and

a calculation section that calculates biological tissue information related to the biological tissue on the basis of a change in intensity of the polarization component of the second polarization direction according to rotation operation performed by the rotation control section.
(2) The observation device according to (1), further including

a detection section that detects, in accordance with the rotation operation, first intensity which is intensity of a polarization component of the second polarization direction extracted by the second polarization section, in which

the calculation section calculates, on the basis of the first intensity detected by the detection section, first intensity data related to a change in first intensity according to the rotation operation.
(3) The observation device according to (2), in which

the calculation section performs a fitting process using a predetermined function on the first intensity data and calculates the biological tissue information on the basis of a process result of the fitting process.
(4) The observation device according to any one of (1) to (3), in which

the biological tissue information includes identification information for identifying whether or not the biological tissue includes an optical anisotropic object.
(5) The observation device according to (4), in which

the biological tissue information includes at least one of first information regarding an orientation direction of the optical anisotropic object or second information regarding orientation and anisotropy of the optical anisotropic object.
(6) The observation device according to (5), in which

the calculation section performs a fitting process using a predetermined periodic function, calculates the first information on the basis of phase information of the predetermined periodic function which is obtained as a process result of the fitting process, and calculates the second information on the basis of amplitude information of the periodic function.
(7) The observation device according to any one of (1) to (6), in which

the detection section generates, in accordance with the rotation operation, an image signal of the biological tissue on the basis of the polarization component of the second polarization direction extracted by the second polarization section and detects the first intensity on the basis of the generated image signal.
(8) The observation device according to (7), in which

the calculation section sets a plurality of target regions, into which an image constituted by the image signal is to be divided, and calculates the biological tissue information with respect to each of the plurality of target regions.
(9) The observation device according to any one of (2) to (8), further including

a third polarization section that extracts the reflection light reflected by the biological tissue while maintaining a polarization state of the reflection light, in which

the detection section detects second intensity which is intensity of the reflection light extracted by the third polarization section.
(10) The observation device according to (9), in which

the rotation control section rotates the first polarization direction by a predetermined angle, and

the calculation section calculates, on the basis of a change in the second intensity according to rotation of the first polarization direction by the predetermined angle, information regarding an orientation direction of an optical anisotropic object which is included in the biological tissue.
(11) The observation device according to (10), in which

the rotation control section rotates the first polarization direction by the predetermined angle on a basis of a predetermined state set on the basis of the change in the first intensity.
(12) The observation device according to (10) or (11), in which

the predetermined angle is ±90°.
(13) The observation device according to any one of (10) to (12), in which

the calculation section determines a quadrant including the orientation direction among quadrants defined by a reference direction that is a reference of the orientation direction and an orthogonal direction orthogonal to the reference direction.
(14) The observation device according to (13), in which

the calculation section calculates an orientation angle between the orientation direction and the reference direction.
(15) The observation device according to any one of (2) to (8), further including

a fourth polarization section that emits non-polarized light to the biological tissue, in which

the detection section detects third intensity that is intensity of a polarization component of the second polarization direction extracted by the second polarization section from beams of the non-polarized light reflected by the biological tissue.
(16) The observation device according to (15), in which

the rotation control section rotates the second polarization direction by a predetermined angle, and

the calculation section calculates, on the basis of a change in the third intensity according to rotation of the second polarization direction by the predetermined angle, information regarding an orientation direction of an optical anisotropic object which is included in the biological tissue.
(17) The observation device according to any one of (1) to (16), in which

the intersection angle is an angle in a range of 90°±2°.
(18) The observation device according to (1) to (17), which is configured as an endoscope or a microscope.
(19) An observation method to be performed by a computer system, the method including:

irradiating a biological tissue with polarization light of a first polarization direction;

extracting a polarization component of a second polarization direction that intersects with the first polarization direction, from beams of reflection light that are the polarization light reflected by the biological tissue;

rotating each of the first polarization direction and the second polarization direction such that an intersection angle between the first polarization direction and the second polarization direction is maintained; and

calculating biological tissue information related to the biological tissue on the basis of a change in intensity of the polarization component of the second polarization direction according to rotation operation of the first polarization direction and the second polarization direction.
(20) A program that causes a computer system to execute:

a step of irradiating a biological tissue with polarization light of a first polarization direction;

a step of extracting a polarization component of a second polarization direction that intersects with the first polarization direction, from beams of reflection light that are the polarization light reflected by the biological tissue;

a step of rotating each of the first polarization direction and the second polarization direction such that an intersection angle between the first polarization direction and the second polarization direction is maintained; and
a step of calculating biological tissue information related to the biological tissue on the basis of a change in intensity of the polarization component of the second polarization direction according to rotation operation of the first polarization direction and the second polarization direction.

### REFERENCE SIGNS LIST

Φ intersection angle
ω rotation angle
Ω, Ω′ predetermined angle
1 observation target
3 polarization light
4, 4a to 4c reflection light
5, 5a, 5b polarization component
20, 220 illumination system
21, 221 light source
22, 222 first polarization element
31, 231 second polarization element
32, 232 image sensor
40, 240 controller
29 first polarization direction
37 second polarization direction
41 rotation control section
42 intensity detection section
43 analysis unit
53 anisotropic object
56 fiber direction
74 ROI
93 even-numbered quadrant
94 odd-numbered quadrant
100, 200 endoscopic device

1. An observation device comprising:
a first polarization section that irradiates a biological tissue with polarization light of a first polarization direction;
a second polarization section that extracts a polarization component of a second polarization direction that intersects with the first polarization direction, from beams of reflection light that are the polarization light reflected by the biological tissue;
a rotation control section that rotates each of the first polarization direction and the second polarization direction such that an intersection angle between the first polarization direction and the second polarization direction is maintained; and
a calculation section that calculates biological tissue information related to the biological tissue on the basis of a change in intensity of the polarization component of the second polarization direction according to rotation operation performed by the rotation control section.

2. The observation device according to claim 1, further comprising
a detection section that detects, in accordance with the rotation operation, first intensity which is intensity of a polarization component of the second polarization direction extracted by the second polarization section, wherein
the calculation section calculates, on the basis of the first intensity detected by the detection section, first intensity data related to a change in first intensity according to the rotation operation.

3. The observation device according to claim 2, wherein
the calculation section performs a fitting process using a predetermined function on the first intensity data and calculates the biological tissue information on the basis of a process result of the fitting process.

4. The observation device according to claim 1, wherein
the biological tissue information includes identification information for identifying whether or not the biological tissue includes an optical anisotropic object.

5. The observation device according to claim 4, wherein
the biological tissue information includes at least one of first information regarding an orientation direction of the optical anisotropic object or second information regarding orientation and anisotropy of the optical anisotropic object.

6. The observation device according to claim 5, wherein
the calculation section performs a fitting process using a predetermined periodic function, calculates the first information on the basis of phase information of the predetermined periodic function which is obtained as a process result of the fitting process, and calculates the second information on the basis of amplitude information of the periodic function.

7. The observation device according to claim 1, wherein
the detection section generates, in accordance with the rotation operation, an image signal of the biological tissue on the basis of the polarization component of the second polarization direction extracted by the second polarization section and detects the first intensity on the basis of the generated image signal.

8. The observation device according to claim 7, wherein
the calculation section sets a plurality of target regions, into which an image constituted by the image signal is to be divided, and calculates the biological tissue information with respect to each of the plurality of target regions.

9. The observation device according to claim 2, further comprising
a third polarization section that extracts the reflection light reflected by the biological tissue while maintaining a polarization state of the reflection light, wherein
the detection section detects second intensity which is intensity of the reflection light extracted by the third polarization section.

10. The observation device according to claim 9, wherein
the rotation control section rotates the first polarization direction by a predetermined angle, and
the calculation section calculates, on the basis of a change in the second intensity according to rotation of the first polarization direction by the predetermined angle, information regarding an orientation direction of an optical anisotropic object which is included in the biological tissue.

11. The observation device according to claim 10, wherein
the rotation control section rotates the first polarization direction by the predetermined angle on a basis of a predetermined state set on the basis of the change in the first intensity.

12. The observation device according to claim 10, wherein the predetermined angle is ±90°.

13. The observation device according to claim 10, wherein
the calculation section determines a quadrant including the orientation direction among quadrants defined by a reference direction that is a reference of the orientation direction and an orthogonal direction orthogonal to the reference direction.

14. The observation device according to claim 13, wherein
the calculation section calculates an orientation angle between the orientation direction and the reference direction.

15. The observation device according to claim 2, further comprising
a fourth polarization section that emits non-polarized light to the biological tissue, wherein
the detection section detects third intensity that is intensity of a polarization component of the second polarization direction extracted by the second polarization section from beams of the non-polarized light reflected by the biological tissue.

16. The observation device according to claim 15, wherein
the rotation control section rotates the second polarization direction by a predetermined angle, and
the calculation section calculates, on the basis of a change in the third intensity according to rotation of the second polarization direction by the predetermined angle, information regarding an orientation direction of an optical anisotropic object which is included in the biological tissue.

17. The observation device according to claim 1, wherein
the intersection angle is an angle in a range of 90°±2°.

18. The observation device according to claim 1, which is configured as an endoscope or a microscope.

19. An observation method to be performed by a computer system, the method comprising:
irradiating a biological tissue with polarization light of a first polarization direction;
extracting a polarization component of a second polarization direction that intersects with the first polarization direction, from beams of reflection light that are the polarization light reflected by the biological tissue;
rotating each of the first polarization direction and the second polarization direction such that an intersection angle between the first polarization direction and the second polarization direction is maintained; and
calculating biological tissue information related to the biological tissue on the basis of a change in intensity of the polarization component of the second polarization direction according to rotation operation of the first polarization direction and the second polarization direction.

20. A program that causes a computer system to execute:
a step of irradiating a biological tissue with polarization light of a first polarization direction;
a step of extracting a polarization component of a second polarization direction that intersects with the first polarization direction, from beams of reflection light that are the polarization light reflected by the biological tissue;
a step of rotating each of the first polarization direction and the second polarization direction such that an intersection angle between the first polarization direction and the second polarization direction is maintained; and
a step of calculating biological tissue information related to the biological tissue on the basis of a change in intensity of the polarization component of the second polarization direction according to rotation operation of the first polarization direction and the second polarization direction.
